(19) United States
(12) Patent Application Publication
(10) Pub. No.: US 2023/0126277 A1
(43) Pub. Date: Apr. 27, 2023

(54) Crystalline forms of Voxelotor, and Processes for the Preparation Thereof

(71) Applicant: JOHNSON MATTHEY PUBLIC LIMITED COMPANY, London (GB)

(72) Inventors: Thierry BONNAUD, Cambridge (GB); Zoe PRENTICE, Cambridge (GB)

(21) Appl. No.: 17/904,889

(22) PCT Filed: Feb. 17, 2021
(86) PCT No.: PCT/GB2021/050380

§ 371 Date: Aug. 24, 2022

(30) Foreign Application Priority Data
Feb. 24, 2020 (GB) 2002560.7

(51) Int. Cl. A61K 47/54

(52) U.S. Cl. CPC A61K 47/542

(57) ABSTRACT

The present invention relates to crystalline forms of voxelotor, to processes for their preparation, and to pharmaceutical compositions containing the crystalline forms.

The present invention relates to crystalline forms of voxelotor, to processes for their preparation, and to pharmaceutical compositions containing the crystalline forms.

### BACKGROUND

Voxelotor has the IUPAC name of 2-hydroxy-6-((2-(1-isopropyl-1H-pyrazol-5-yl)pyridin-3-yl)methoxy)benzaldehyde or 2-hydroxy-6-[[2-(2-propan-2-ylpyrazol-3-yl)pyridin-3-yl]methoxy]benzaldehyde and has the chemical structure illustrated below:

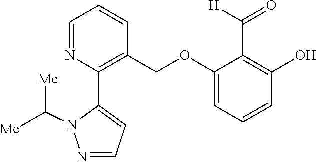 [SMILES: CC(C)n1nccc1-c1ncccc1COc1cccc(O)c1C=O]

EP2797416B and EP3141542A (to Global Blood Therapeutics) describe voxelotor and its preparation.

In the EU, an orphan designation has been granted to voxelotor for the treatment of sickle cell disease.

Information about the solid-state properties of a drug substance is important. For example, different forms may have differing solubilities. Also, the handling and stability of a drug substance may depend on the solid form.

Polymorphism may be defined as the ability of a compound to crystallise in more than one distinct crystal species and different crystal arrangements of the same chemical composition are termed polymorphs. Polymorphs of the same compound arise due to differences in the internal arrangement of atoms and have different free energies and therefore different physical properties such as solubility, chemical stability, melting point, density, flow properties, hygroscopicity, bioavailability, and so forth. The compound voxelotor may exist in a number of polymorphic forms and many of these forms may be undesirable for producing pharmaceutically acceptable compositions. This may be for a variety of reasons including lack of stability, high hygroscopicity, low aqueous solubility and difficulty in handing.

### Definitions

The term “about” or “approximately” means an acceptable error for a particular value as determined by a person of ordinary skill in the art, which depends in part on how the value is measured or determined. In certain embodiments, the term “about” or “approximately” means within 1, 2, 3 or 4 standard deviations. In certain embodiments, the term “about” or “approximately” means within 30%, 25%, 20%, 15%, 10%, 9%, 8%, 7%, 6%, 5%, 4%, 3%, 2%, 1%, or 0.5% of a given value or range. In certain embodiments and with reference to X-ray powder diffraction two-theta peaks, the terms “about” or “approximately” means within ±0.2° 20.

The term “ambient temperature” means one or more room temperatures between about 15° C. to about 30° C., such as about 15° C. to about 25° C.

The term “anti-solvent” refers to a first solvent which is added to a second solvent to reduce the solubility of a compound in that second solvent. The solubility may be reduced sufficiently such that precipitation of the compound from the first and second solvent combination occurs.

The term “consisting” is closed and excludes additional, unrecited elements or method steps in the claimed invention.

The term “consisting essentially of” is semi-closed and occupies a middle ground between “consisting” and “comprising”. “Consisting essentially of” does not exclude additional, unrecited elements or method steps which do not materially affect the essential characteristic(s) of the claimed invention.

The term “comprising” is inclusive or open-ended and does not exclude additional, unrecited elements or method steps in the claimed invention. The term is synonymous with “including but not limited to”. The term “comprising” encompasses three alternatives, namely (i) “comprising”, (ii) “consisting”, and (iii) “consisting essentially of”.

The term “crystalline” and related terms used herein, when used to describe a compound, substance, modification, material, component or product, unless otherwise specified, means that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, Md. (2005); The United States Pharmacopeia, 23rd ed., 1843-1844 (1995).

The term “molecular complex” is used to denote a crystalline material composed of two or more different components which has a defined single-phase crystal structure. The components are held together by non-covalent bonding, such as hydrogen bonding, ionic bonding, van der Waals interactions, pi-pi interactions, etc. The term “molecular complex” includes salts, co-crystals and salt/co-crystal hybrids.

In one embodiment, the molecular complex is a co-crystal. Without wishing to be bound by theory, it is believed that when the molecular complex is a co-crystal, the co-crystal demonstrates improved physiochemical properties, such as crystallinity, solubility properties and/or modified melting points.

The terms “polymorph,” “polymorphic form” or related term herein, refer to a crystal form of one or more molecules of voxelotor, or voxelotor molecular complex thereof that can exist in two or more forms, as a result different arrangements or conformations of the molecule(s) in the crystal lattice of the polymorph.

The term “pharmaceutical composition” is intended to encompass a pharmaceutically effective amount of voxelotor of the invention and a pharmaceutically acceptable excipient. As used herein, the term “pharmaceutical compositions” includes pharmaceutical compositions such as tablets, pills, powders, liquids, suspensions, emulsions, granules, capsules, suppositories, or injection preparations.

The term “excipient” refers to a pharmaceutically acceptable organic or inorganic carrier substance. Excipients may be natural or synthetic substances formulated alongside the active ingredient of a medication, included for the purpose of bulking-up formulations that contain potent active ingredients (thus often referred to as “bulking agents,” “fillers,” or “diluents”), or to confer a therapeutic enhancement on the active ingredient in the final dosage form, such as facilitating drug absorption or solubility. Excipients can also be useful in the manufacturing process, to aid in the handling of the active substance, such as by facilitating powder flowability or non-stick properties, in addition to aiding in vitro stability such as prevention of denaturation over the expected shelf life.

The term “patient” refers to an animal, preferably a patient, most preferably a human, who has been the object of treatment, observation or experiment. Preferably, the patient has experienced and/or exhibited at least one symptom of the disease or disorder to be treated and/or prevented. Further, a patient may not have exhibited any symptoms of the disorder, disease or condition to be treated and/prevented, but has been deemed by a physician, clinician or other medical professional to be at risk for developing said disorder, disease or condition.

The term “solvate” refers to a combination or aggregate formed by one or more molecules of a solute e.g. voxelotor, and one or more molecules of a solvent. The one or more molecules of the solvent may be present in stoichiometric or non-stoichiometric amounts to the one or more molecules of the solute.

The terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more therapeutic agents to a patient with such a disease or disorder. In some embodiments, the terms refer to the administration of a molecular complex provided herein, with or without other additional active agents, after the onset of symptoms of a disease.

The term “overnight” refers to the period of time between the end of one working day to the subsequent working day in which a time frame of about 12 to about 18 hours has elapsed between the end of one procedural step and the instigation of the following step in a procedure.

### BRIEF DESCRIPTION OF THE FIGURES

Certain aspects of the embodiments described herein may be more clearly understood by reference to the drawings, which are intended to illustrate but not limit, the invention, and wherein:

FIG. 1 is a representative XRPD pattern of voxelotor hemi propylene glycol solvate.

FIG. 2 is a representative TGA thermogram and a DSC thermogram of voxelotor hemi propylene glycol solvate.

FIG. 3 is a representative XRPD pattern of voxelotor hemi fumaric acid molecular complex.

FIG. 4 is a representative TGA thermogram and a DSC thermogram of voxelotor hemi fumaric acid molecular complex.

FIG. 5 is a representative XRPD pattern of voxelotor hemi succinic acid molecular complex.

FIG. 6 is a representative TGA thermogram and a DSC thermogram of voxelotor hemi succinic acid molecular complex.

FIG. 7 illustrates how centrifugal forces are applied to particles in the Speedmixer™. FIG. 7A is a view from above showing the base plate and basket. The base plate rotates in a clockwise direction.

FIG. 7B is a side view of the base plate and basket.

FIG. 7C is a view from above along line A in FIG. 7B. The basket rotates in an anti-clockwise direction.

### DESCRIPTION OF THE INVENTION

Voxelotor Hemi Propylene Glycol Solvate

It has been discovered that voxelotor can be prepared in a well-defined and consistently reproducible propylene glycol solvate form. Moreover, a reliable and scalable method for producing this solvate form has been developed. The voxelotor polymorph provided by the present invention may be useful as an active ingredient in pharmaceutical formulations. In certain embodiments, the crystalline solvate form is purifiable. In certain embodiments and depending on time, temperature and humidity, the crystalline solvate form is stable. In certain embodiments, the crystalline solvate form is easy to isolate and handle. In certain embodiments, the process for preparing the crystalline solvate form is scalable.

The crystalline form described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In one aspect, the present invention provides a crystalline form of voxelotor which is crystalline voxelotor hemi propylene glycol solvate.

The molar ratio of voxelotor to propylene glycol may be in the range of about 1 mole of voxelotor:about 0.3 to about 1 moles of propylene glycol, for example about 1 mole of voxelotor:about 0.4 to about 0.7 moles of propylene glycol. In one embodiment, the molar ratio of voxelotor to propylene glycol may be about 1 mole of voxelotor:about 0.5 moles of propylene glycol.

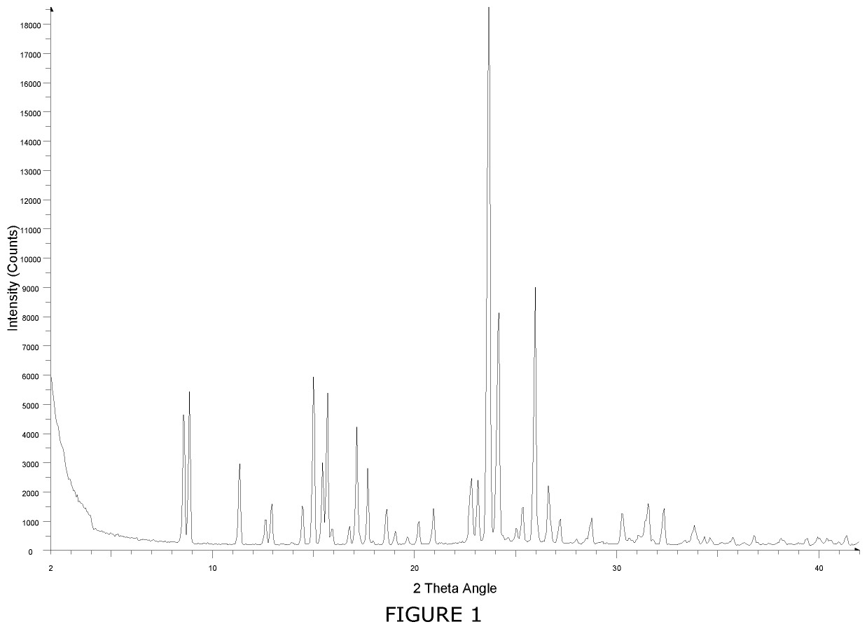

The hemi propylene glycol solvate may have an X-ray powder diffraction pattern comprising one or more peaks (for example 1, 2, 3, 4, 5, 6, 7, 8, 9, or 10 peaks) selected from the group consisting of about 8.6, 8.8, 11.3, 12.6, 12.9, 14.5, 15.0, 15.5, 15.6, 16.0, 16.8, 17.1, 17.7, 18.0, 18.6, 19.1, 19.7, 20.2, 20.9, 22.8, 23.1, 23.7, 24.2, 25.1, 25.4, 25.9, 26.7, 27.2, 28.8, 30.3, 31.6, and 32.4 degrees two-theta±0.2 degrees two-theta. In one embodiment, the solvate may have the X-ray powder diffraction pattern substantially as shown in FIG. 1.

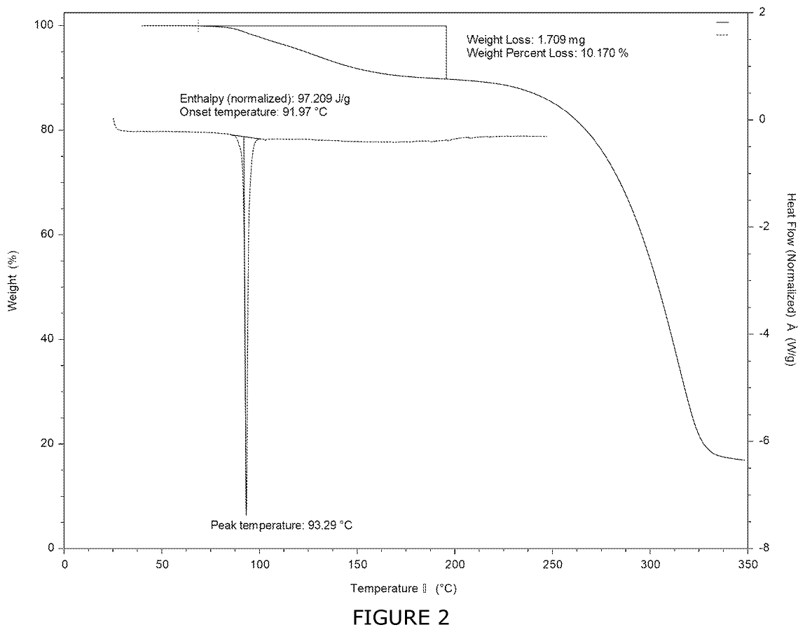

The hemi propylene glycol solvate may have a DSC thermogram comprising an endothermic event with an onset temperature of about 92.0° C. In one embodiment, the solvate may have a DSC thermogram substantially as shown in FIG. 2.

The hemi propylene glycol solvate may have a TGA thermogram comprising about 10.2% mass loss when heated from about ambient temperature to about 200° C. In one embodiment, the solvate may have a TGA thermogram substantially as shown in FIG. 2.

The crystalline voxelotor hemi propylene glycol solvate formed may be free or substantially free of other polymorphic forms of voxelotor. In certain embodiments, the polymorphic purity of the solvate is ≥90%, ≥91%, ≥92%, ≥93%, ≥94%, ≥95% or higher. In certain embodiments, the polymorphic purity of the solvate is ≥95%. In certain embodiments, the polymorphic purity of the solvate is ≥96%. In certain embodiments, the polymorphic purity of the solvate is ≥97%. In certain embodiments, the polymorphic purity of the solvate is ≥98%. In certain embodiments, the polymorphic purity of the solvate is ≥99%.

The crystalline voxelotor hemi propylene glycol solvate described above may be prepared by a process comprising reacting voxelotor and propylene glycol using low energy ball milling or low energy grinding.

Propylene glycol is present in sufficient quantities to form the desired solvate. The quantity of propylene glycol is not particularly limiting provided there is enough propylene glycol to dissolve the voxelotor and form a solution, suspend the voxelotor, or wet the voxelotor. In one embodiment, the w/v ratio of voxelotor to propylene glycol may be in the range from about 1 mg of voxelotor:about 0.01 to about 1.5 μl propylene glycol, such as about 1 mg of voxelotor:about 0.05 to about 1.0 μl propylene glycol, for example about 1 mg of voxelotor:about 0.1 to about 0.75 μl propylene glycol, e.g. about 1 mg of voxelotor:about 0.5 μl propylene glycol.

When low energy ball milling is utilised, the milling process may be controlled by various parameters including the speed at which the milling takes place, the length of milling time and/or the level to which the milling container is filled.

The speed at which the milling takes place may be from about 50 rpm to about 1000 rpm. In one embodiment, the speed may be from about 75 rpm to about 750 rpm. In another embodiment, the speed may be from about 80 rpm to about 650 rpm. In one embodiment, the speed may be about 500 rpm.

Low energy grinding involves shaking the materials within a grinding container. The grinding occurs via the impact and friction of the materials within the container. The process may be controlled by various parameters including the frequency at which the grinding takes place, the length of grinding time and/or the level to which the container is filled.

The frequency at which the grinding takes place may be from about 1 Hz to about 100 Hz. In one embodiment, the frequency may be from about 10 Hz to about 70 Hz. In another embodiment, the frequency may be from about 20 Hz to about 50 Hz. In one embodiment, the frequency may be about 30 Hz.

Regardless of whether milling or grinding is used, milling or grinding media may be used to assist the reaction. In this instance, the incorporation of hard, non-contaminating media can additionally assist in the breakdown of particles where agglomeration has occurred, for example, as a result of the manufacturing process or during transit. Such breakdown of the agglomerates further enhances the reaction of voxelotor with propylene glycol. The use of milling/grinding media is well-known within the field of powder processing and materials such as stabilised zirconia and other ceramics are suitable provided they are sufficiently hard or ball bearings e.g. stainless steel ball bearings.

Regardless of whether milling or grinding is used, an improvement in the process can be made by controlling the particle ratio, the size of the milling/grinding media and other parameters as are familiar to the skilled person.

The length of milling or grinding time may be from about 1 minute to about 2 days, for example, about 10 minutes to about 5 hours, such as about 20 minutes to 3 hours, e.g. about 2 hours.

The voxelotor and propylene glycol may be contacted at ambient temperature or less. Alternatively, the voxelotor may be contacted with the propylene glycol at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa).

The voxelotor hemi propylene glycol solvate is recovered as a crystalline solid. The crystalline solvate may be recovered by directly by filtering, decanting or centrifuging. If desired, a proportion of the propylene glycol may be evaporated prior to recovery of the crystalline solid.

Alternatively, the voxelotor hemi propylene glycol solvate described above may be prepared by a process comprising the step of applying dual asymmetric centrifugal forces to a mixture of voxelotor and propylene glycol to form the solvate.

Propylene glycol is present in sufficient quantities to form the desired solvate. The quantity of propylene glycol is not particularly limiting provided there is enough propylene glycol to dissolve the voxelotor and form a solution, suspend the voxelotor, or moisten the voxelotor. In one embodiment, the w/v ratio of voxelotor to propylene glycol may be in the range from about 1 mg of voxelotor:about 0.01 to about 1.5 μl propylene glycol, such as about 1 mg of voxelotor: about 0.05 to about 1.0 μl propylene glycol, for example about 1 mg of voxelotor:about 0.1 to about 0.75 μl propylene glycol, e.g. about 1 mg of voxelotor:about 0.5 μl propylene glycol.

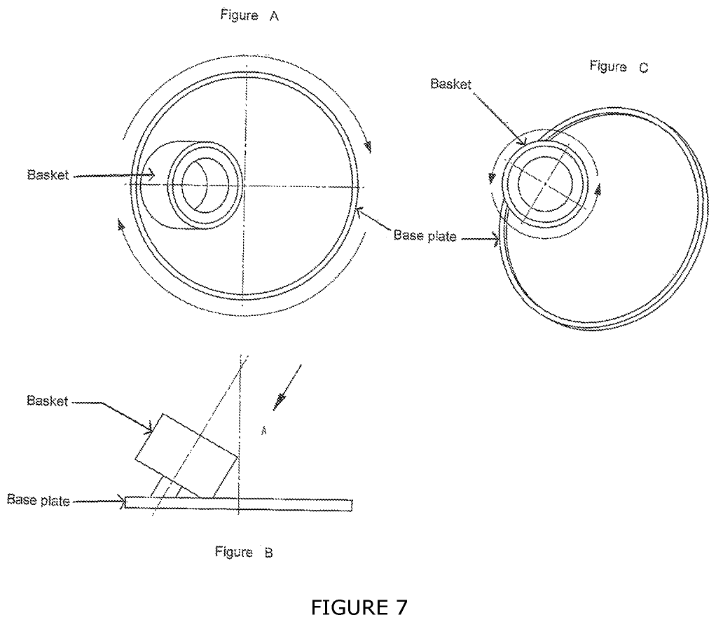

The voxelotor hemi propylene glycol solvate is formed using dual asymmetric centrifugal forces. By “dual asymmetric centrifugal forces” we mean that two centrifugal forces, at an angle to each other, are simultaneously applied to the particles. In order to create an efficient mixing environment, the centrifugal forces preferably rotate in opposite directions. The Speedmixer™ by Hauschild (http://www.speedmixer.co.uk/index.php) utilises this dual rotation method whereby the motor of the Speedmixer™ rotates the base plate of the mixing unit in a clockwise direction (see FIG. 7A) and the basket is spun in an anti-clockwise direction (see FIGS. 7B and 7C).

The process may be controlled by various parameters including the rotation speed at which the process takes place, the length of processing time, the level to which the mixing container is filled, the use of milling media and/or the control of the temperature of the components within the milling pot.

The dual asymmetric centrifugal forces may be applied for a continuous period of time. By “continuous” we mean a period of time without interruption. The period of time may be from about 1 second to about 10 minutes, such as about 5 seconds to about 5 minutes, for example, about 10 seconds to about 200 seconds e.g. 2 minutes.

Alternatively, the dual asymmetric centrifugal forces may be applied for an aggregate period of time. By “aggregate” we mean the sum or total of more than one periods of time (e.g. 2, 3, 4, 5 or more times). The advantage of applying the centrifugal forces in a stepwise manner is that excessive heating of the particles can be avoided. The dual asymmetric centrifugal forces may be applied for an aggregate period of about 1 second to about 20 minutes, for example about 30 seconds to about 15 minutes and such as about 10 seconds to about 10 minutes e.g. 6 minutes. In one embodiment, the dual asymmetric centrifugal forces are applied in a stepwise manner with periods of cooling therebetween. In another embodiment, the dual asymmetric centrifugal forces may be applied in a stepwise manner at one or more different speeds.

The speed of the dual asymmetric centrifugal forces may be from about 200 rpm to about 4000 rpm. In one embodiment, the speed may be from about 300 rpm to about 3750 rpm, for example about 500 rpm to about 3500 rpm. In one embodiment, the speed may be about 3500 rpm. In another embodiment, the speed may be about 2300 rpm.

The level to which the mixing container is filled is determined by various factors which will be apparent to the skilled person. These factors include the apparent density of the voxelotor and propylene glycol, the volume of the mixing container and the weight restrictions imposed on the mixer itself.

Milling media as described above may be used to assist the reaction. In certain embodiments, the dual asymmetric centrifugal forces may be applied in a stepwise manner in which milling media may be used for some, but not all, periods of time.

The voxelotor hemi propylene glycol solvate is recovered as a crystalline solid. The crystalline solvate may be recovered by directly by filtering, decanting or centrifuging. If desired, a proportion of the propylene glycol may be evaporated prior to recovery of the crystalline solid.

Howsoever the crystalline solvate is recovered, the separated solvate may be dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline solvate may be left to dry under ambient temperature naturally i.e. without the active application of vacuum. It is preferred that the drying conditions are maintained below the point at which the solvate degrades and so when the solvate is known to degrade within the temperature or pressure ranges given above, the drying conditions should be maintained below the degradation temperature or vacuum.

The crystalline voxelotor hemi propylene glycol solvate described above may be prepared by a process comprising the steps of:
(a) contacting voxelotor with a first solvent selected from the group consisting of tert-butyl methyl ether (TBME), isopropyl acetate, diethyl ether, 2-methyl tetrahydrofuran (2-methyl THF), and combinations thereof; and
(b) adding propylene glycol to the solution or suspension of voxelotor; and
(c) recovering voxelotor hemi propylene glycol solvate as a crystalline solid.

The quantity of the first solvent is not particularly limiting provided there is enough solvent to dissolve the voxelotor and form a solution, or suspend the voxelotor. The w/v ratio of voxelotor to the first solvent may be in the range of about 1 mg of voxelotor:about 1 to about 1000 μl of solvent, such as about 1 mg of voxelotor:about 1 to about 500 μl of solvent, for example about 1 mg of voxelotor:about 1 to about 150 μl of solvent, e.g. about 1 mg of voxelotor:about 1 to about 10 μl of solvent. In one embodiment, the w/v ratio of voxelotor to the first solvent may be about 1 mg of voxelotor:about 4 μl of solvent.

The voxelotor may be contacted with the first solvent at ambient temperature or less. In one embodiment, the contacting step may be carried out at one or more temperatures in the range of ≥about 0° C. to about ≤25° C. In some embodiments, the contacting step is carried out at one or more temperatures≥about 1° C. In some embodiments, the contacting step is carried out at one or more temperatures≥about 2° C. In some embodiments, the contacting step is carried out at one or more temperatures≥about 3° C. In some embodiments, the contacting step is carried out at one or more temperatures≥about 4° C. In some embodiments, the contacting step is carried out at one or more temperatures≥about 5° C. In some embodiments, the contacting step is carried out at one or more temperatures≤about 20° C. In some embodiments, the contacting step is carried out at one or more temperatures≤about 15° C. In some embodiments, the contacting step is carried out at one or more temperatures≤about 10° C. In one embodiment, the contacting step is carried out at one or more temperatures in the range of ≥about 0° C. to ≤about 10° C., for example, about 5° C. In one embodiment, the contacting step may be carried out at about ambient temperature e.g. about 25° C.

Alternatively, the voxelotor may be contacted with the solvent at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa). In one embodiment, the contacting step may be carried out at one or more temperatures in the range of ≥about 40° C. to ≤about 60° C. In some embodiments, the contacting step is carried out at one or more temperatures≥about 41° C. In some embodiments, the contacting step is carried out at one or more temperatures≥about 42° C. In some embodiments, the contacting step is carried out at one or more temperatures≥about 43° C. In some embodiments, the contacting step is carried out at one or more temperatures≥about 44° C. In some embodiments, the contacting step is carried out at one or more temperatures≥about 45° C. In some embodiments, the contacting step is carried out at one or more temperatures≥about 46° C. In some embodiments, the contacting step is carried out at one or more temperatures≥about 47° C. In some embodiments, the contacting step is carried out at one or more temperatures≥about 48° C. In some embodiments, the contacting step is carried out at one or more temperatures≥about 49° C. In some embodiments, the contacting step is carried out at one or more temperatures≥about 50° C.

In some embodiments, the contacting step is carried out at one or more temperatures≤about 59° C. In some embodiments, the contacting step is carried out at one or more temperatures≤about 58° C. In some embodiments, the contacting step is carried out at one or more temperatures≤about 57° C. In some embodiments, the contacting step is carried out at one or more temperatures≤about 56° C. In some embodiments, the contacting step is carried out at one or more temperatures≤about 55° C. In some embodiments, the contacting step is carried out at one or more temperatures≤about 54° C. In some embodiments, the contacting step is carried out at one or more temperatures≤about 53° C. In some embodiments, the contacting step is carried out at one or more temperatures≤about 52° C. In some embodiments, the contacting step is carried out at one or more temperatures≤about 51° C. In one embodiment, the contacting step is carried out at one or more temperatures in the range of ≥about 45° C. to ≥about 55° C. In one embodiment, the contacting step is carried out at a temperature of about 50° C.

The dissolution or suspension of voxelotor may be encouraged through the use of an aid such as stirring, shaking and/or sonication. Additional solvent may be added to aid the dissolution or suspension of the voxelotor.

The period of time for which the mixture of voxelotor and solvent is treated at the desired temperature is not particularly limiting. In one embodiment, the period of time may be from about 1 minute to about 24 hours, for example, about 5 minutes.

In step (b), propylene glycol is added to the reaction mixture. The quantity of propylene glycol is not particularly limiting. In one embodiment, the w/v ratio of voxelotor to propylene glycol may be in the range from about 1 mg of voxelotor:about 0.01 to about 1.5 μl propylene glycol, such as about 1 mg of voxelotor:about 0.05 to about 1.0 μl propylene glycol, for example about 1 mg of voxelotor:about 0.1 to about 0.75 μl propylene glycol, e.g. about 1 mg of voxelotor:about 0.1 μl to about 0.4 μl propylene glycol. These w/v ratios have been calculated using the mass of voxelotor initially dissolved or suspended in the first solvent i.e. the quantity of voxelotor inputted into the process.

After the addition of the propylene glycol, the reaction mixture may be treated for a period of time at ambient temperature or less as described above in connection with first solvent.

Alternatively, the reaction mixture may be treated for a period of time at one or more temperatures greater than ambient i.e. greater than 30° C. and below the boiling point of the reaction mixture as described above in connection with the first solvent.

The reaction mixture may be left for a further period of time, e.g. about 1 minute to about 24 hours, such as about 1 hour.

The solution or suspension may then be cooled such that the resulting solution or suspension has a temperature below that of the solution or suspension of step (b). The rate of cooling may be from about 0.05° C./minute to about 2° C./minute, such as about 0.1° C./minute to about 1.5° C./minute, for example about 0.1° C./minute or 0.5° C./minute. When a solution of voxelotor and propylene glycol is cooled, a suspension may eventually be observed.

The solution or suspension may be cooled to ambient temperature or a temperature of less than ambient temperature. In one embodiment, the solution or suspension may be cooled to one or more temperatures in the range of ≥about 0° C. to ≤about 20° C. In some embodiments, the solution or suspension is cooled to one or more temperatures≥about 1° C. In some embodiments, the solution or suspension is cooled to one or more temperatures≥about 2° C. In some embodiments, the solution or suspension is cooled to one or more temperatures≥about 3° C. In some embodiments, the solution or suspension is cooled to one or more temperatures≥about 4° C. In some embodiments, the solution or suspension is cooled to one or more temperatures≥about 5° C. In some embodiments, the solution or suspension is cooled to one or more temperatures≤about 15° C. In some embodiments, the solution or suspension is cooled to one or more temperatures≤about 14° C. In some embodiments, the solution or suspension is cooled to one or more temperatures≤about 13° C. In some embodiments, the solution or suspension is cooled to one or more temperatures≤about 12° C. In some embodiments, the solution or suspension may be cooled to one or more temperatures≤about 11° C. In some embodiments, the solution or suspension is cooled to one or more temperatures≤about 10° C. In one embodiment, the solution or suspension is cooled to one or more temperatures in the range of about 5° C. to about 10° C.

In certain embodiments, an anti-solvent may be added to the solution or suspension after the solution or suspension has been cooled to one or more temperatures less than ambient temperature as described above. The anti-solvent may be pre-cooled to a suitable temperature before it is added to the cooled solution or suspension. In one embodiment, the anti-solvent is an alkane solvent, such as heptane. In one embodiment, the anti-solvent is heptane and it is added to the solution or suspension of voxelotor hemi propylene glycol solvate at about 15° C. After addition of the anti-solvent, cooling may continue as described above.

In step (c), the voxelotor hemi propylene glycol solvate is recovered as a crystalline solid. The crystalline solvate may be recovered by directly by filtering, decanting or centrifuging. If desired, the suspension may be mobilised with additional portions of the solvent prior to recovery of the crystalline solid. Alternatively, a proportion or substantially all of the solvent may be evaporated prior to recovery of the crystalline solid.

Howsoever the crystalline solvate is recovered, the separated solvate may be washed with solvent (e.g. one or more of the solvents described above) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline solvate may be left to dry under ambient temperature naturally i.e. without the active application of vacuum. It is preferred that the drying conditions are maintained below the point at which the solvate degrades and so when the solvate is known to degrade within the temperature or pressure ranges given above, the drying conditions should be maintained below the degradation temperature or vacuum.

Steps (a) to (c) may be carried out one or more times (e.g. 1, 2, 3, 4 or 5 times). When steps (a) to (c) are carried out more than once (e.g. 2, 3, 4 or 5 times), step (a) may be optionally seeded with crystalline voxelotor hemi propylene glycol solvate (which was previously prepared and isolated by a method described herein).

Alternatively or in addition, when steps (a) to (c) are carried out more than once (e.g. 2, 3, 4 or 5 times), the solution or suspension formed in step (b) may be optionally seeded with crystalline voxelotor hemi propylene glycol solvate (which was previously prepared and isolated by a method described herein).

The inventors envisage that the voxelotor hemi propylene glycol solvate described above may be prepared by a process comprising the steps of:

(a) providing an admixture of voxelotor and propylene glycol; and

(b) feeding the admixture through an extruder to form the voxelotor hemi propylene glycol solvate.

The admixture is a blend of voxelotor and propylene glycol. The admixture may be prepared by mixing voxelotor and propylene glycol by any suitable means, e.g. by using a tubular blender, for a suitable period of time e.g. about 30 minutes. It is desirable but not essential to prepare a homogeneous blend of voxelotor and propylene glycol.

The propylene glycol may be present in stoichiometric or excess molar equivalents to the voxelotor. In one embodiment, the propylene glycol is present in stoichiometric quantities. The molar ratio of voxelotor to propylene glycol may be in the range of about 1 mole of voxelotor:about 0.3 to about 1 moles of propylene glycol, for example about 1 mole of voxelotor:about 0.4 to about 0.7 moles of propylene glycol. In one embodiment, the molar ratio of voxelotor to propylene glycol may be about 1 mole of voxelotor:about 0.5 moles of propylene glycol.

The solvate does not form on preparing the admixture. The voxelotor and propylene glycol form the solvate as the admixture is processed through the extruder.

An extruder typically includes a rotating screw or screws within a stationary barrel with a die located at one end of the barrel. Along the entire length of the screw, the solvation of the admixture is provided by the rotation of the screw(s) within the barrel. The extruder can be divided into at least three sections: a feeding section; a heating section and a metering section. In the feeding section, the admixture is fed into the extruder. The admixture can be directly added to the feeding section with or without the need of a solvent. In the heating section, the admixture is heated to a temperature such that the voxelotor and propylene glycol solvate to form the voxelotor hemi propylene glycol solvate as the admixture transverses the section. A solvent may be optionally added in the heating section. After the heating section is an optional metering section in which the solvate may be extruded through a die into a particular shape, e.g., granules. The extruder may be a single screw extruder, a twin screw extruder, a multi screw extruder or an intermeshing screw extruder. In one embodiment, the extruder is a twin screw extruder e.g. a co-rotating twin screw extruder.

The admixture may be fed into the feeding section at any suitable speed. For example, the speed of the feeding section may be from about 1 rpm to about 100 rpm. In one embodiment, the speed may be from about 5 rpm to about 80 rpm.

In certain embodiments, solvent is added to the admixture as the admixture is fed into the feeding section. Alternatively or in addition, a solvent may be added one or more times (e.g. 1, 2, 3, 4, or 5 times) in one or more zones (e.g. 1, 2, 3, 4, or 5 zones) of the heating section as the admixture traverses the heating section. This may be advantageous in preventing the admixture drying out as the material moves through the heating section.

The quantity of solvent added is not particularly limiting provided sufficient solvent is added to moisten (i.e. “wet”) the admixture but not so large a quantity that the admixture becomes too liquid.

The heating section may be heated to a single temperature across its length or it may be divided into more than one (e.g. 2, 3, 4, or 5) zones, each of which may be heated independently of the other zones. The temperature of the heating section or each zone is not particularly limiting provided that on exiting the heating section the voxelotor and propylene glycol have solvated to form voxelotor hemi propylene glycol solvate and none of voxelotor, propylene glycol and/or the solvate have substantially degraded or substantially decomposed.

When the extruder comprises screws, the screw (or screws) and the heating section may coincide i.e. the screw (or screws) may also be the heating section.

The speed at which the screw (or screws) rotate may be any suitable speed. For example, the speed of the screw (or screws) may be from about 1 rpm to about 500 rpm. In one embodiment, the speed may be from about 5 rpm to about 400 rpm, such as about 10 rpm to about 100 rpm.

The voxelotor hemi propylene glycol solvate is recovered as a crystalline solid. The crystalline molecular complex may be recovered by simply collecting the crystalline product. If desired, a proportion of the solvent (if present) may be evaporated prior to recovery of the crystalline solid.

Howsoever the crystalline molecular complex is recovered, the separated molecular complex may be dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline solvate may be left to dry under ambient temperature naturally i.e. without the active application of vacuum. It is preferred that the drying conditions are maintained below the point at which the solvate degrades and so when the solvate is known to degrade within the temperature or pressure ranges given above, the drying conditions should be maintained below the degradation temperature or vacuum.

In another aspect, the present invention relates to a pharmaceutical composition comprising crystalline voxelotor hemi propylene glycol solvate as described herein and a pharmaceutically acceptable excipient.

In another aspect, the present invention relates to a method for treating a condition associated with oxygen deficiency in a patient comprising administering a therapeutically effective amount of crystalline voxelotor hemi propylene glycol solvate as described herein to the patient. The condition associated with oxygen deficiency may be sickle cell disease.

In another aspect, the present invention relates to crystalline voxelotor hemi propylene glycol solvate as described herein for use in treating a condition associated with oxygen deficiency. The condition associated with oxygen deficiency may be sickle cell disease.

Voxelotor Hemi Fumaric Acid Molecular Complex

It has been discovered that voxelotor can be prepared in a well-defined and consistently reproducible fumaric acid molecular complex. Moreover, a reliable and scalable method for producing this molecular complex has been developed. The voxelotor molecular complex provided by the present invention may be useful as an active ingredient in pharmaceutical formulations. In certain embodiments, the crystalline molecular complex is purifiable. In certain embodiments and depending on time, temperature and humidity, the crystalline molecular complex is stable. In certain embodiments, the crystalline molecular complex is easy to isolate and handle. In certain embodiments, the process for preparing the crystalline molecular complex is scalable.

The crystalline molecular complex described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In another aspect, the present invention provides a crystalline molecular complex of voxelotor and fumaric acid. In one embodiment, the crystalline molecular complex is voxelotor hemi fumaric acid molecular complex e.g. voxelotor hemi fumaric acid co-crystal.

The molar ratio of voxelotor to fumaric acid may be in the range of about 1 mole of voxelotor:about 0.3 to about 1 moles of fumaric acid, for example about 1 mole of voxelotor:about 0.4 to about 0.7 moles of fumaric acid. In one embodiment, the molar ratio of voxelotor to fumaric acid may be about 1 mole of voxelotor:about 0.5 moles of fumaric acid.

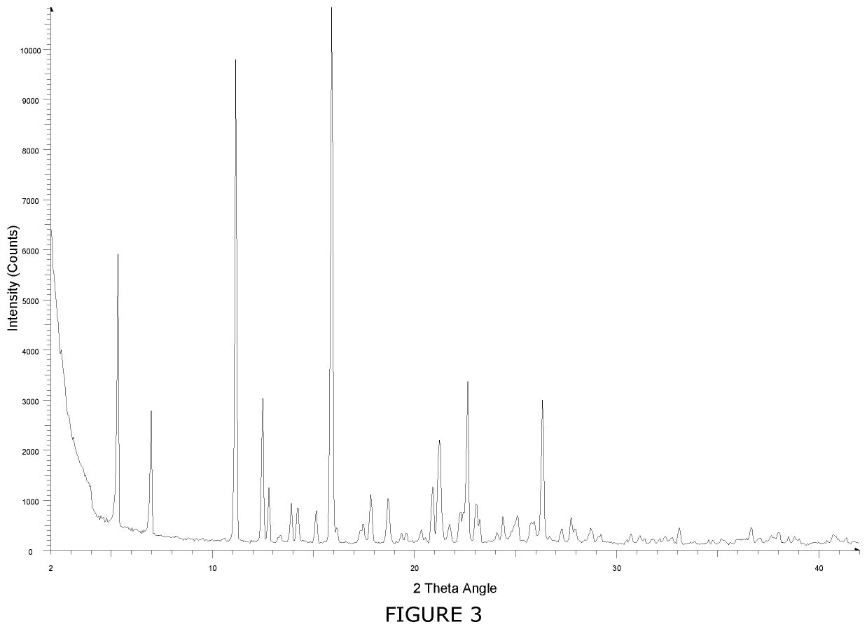

The hemi fumaric acid molecular complex may have an X-ray powder diffraction pattern comprising one or more peaks (for example 1, 2, 3, 4, 5, 6, 7, 8, 9, or 10 peaks) selected from the group consisting of about 5.3, 6.9, 11.2, 12.5, 12.8, 13.4, 13.9, 14.2, 15.1, 15.9, 16.2, 17.3, 17.5, 17.8, 18.7, 19.4, 19.6, 20.3, 20.9, 21.2, 21.7, 22.3, 22.6, 23.1, 23.3, 24.1, 24.4, 24.8, 25.1, 25.8, 25.9, 26.4, and 27.7 degrees two-theta±0.2 degrees two-theta. In one embodiment, the molecular complex may have the X-ray powder diffraction pattern substantially as shown in FIG. 3.

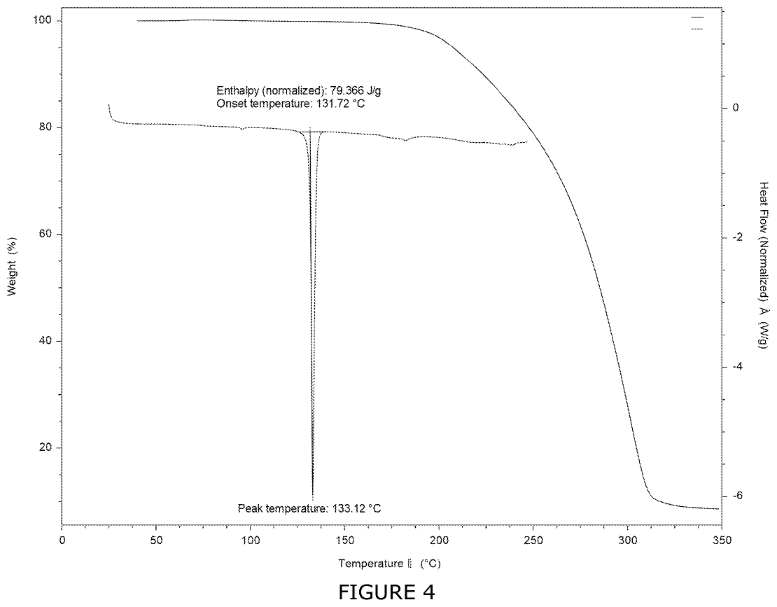

The hemi fumaric acid molecular complex may have a DSC thermogram comprising an endothermic event with an onset temperature of about 131.7° C. In one embodiment, the molecular complex may have a DSC thermogram substantially as shown in FIG. 4.

The hemi fumaric acid molecular complex may have a TGA thermogram comprising no substantial mass loss when heated from about ambient temperature to about 150° C. In one embodiment, the molecular complex may have a TGA thermogram substantially as shown in FIG. 4.

Thermal analysis of the hemi fumaric acid molecular complex shows that there is no loss of fumaric acid immediately after the melt of the solid by TGA. This shows there is a temperature window between the melt of the molecular complex (DSC event at about 131.7° C.) and sample degradation (about 160° C. by TGA), where the liquid can be cooled to reform the molecular complex. This indicates that thermal methods (such as hot melt extrusion) can be used to produce the molecular complex.

The crystalline voxelotor hemi fumaric acid molecular complex formed may be free or substantially free of other polymorphic forms of voxelotor. In certain embodiments, the polymorphic purity of the molecular complex is ≥90%, ≥91%, ≥92%, ≥93%, ≥94%, ≥95% or higher. In certain embodiments, the polymorphic purity of the molecular complex is ≥95%. In certain embodiments, the polymorphic purity of the molecular complex is ≥96%. In certain embodiments, the polymorphic purity of the molecular complex is ≥97%. In certain embodiments, the polymorphic purity of the molecular complex is ≥98%. In certain embodiments, the polymorphic purity of the molecular complex is ≥99%.

The crystalline voxelotor hemi fumaric acid molecular complex described above may be prepared by a process comprising the steps of:
(a) contacting voxelotor and fumaric acid with a first solvent selected from the group consisting of methanol and tert-butyl methyl ether (TMBE), and combinations thereof; and
(b) recovering voxelotor hemi fumaric acid molecular complex as a crystalline solid.

Fumaric acid may be utilised as a solid, or as a solution in a solvent (e.g. methanol and/or TBME).

In one embodiment, step (a) may comprise the steps of:
(a1) contacting voxelotor with a first solvent selected from the group consisting of methanol and tert-butyl methyl ether (TMBE), and combinations thereof; and
(a2) adding fumaric acid to the solution or suspension of voxelotor.

In another embodiment, step (a) may comprise the step of:
(a1′) contacting a solid admixture of voxelotor and fumaric acid with a first solvent selected from the group consisting of methanol and tert-butyl methyl ether (TMBE), and combinations thereof to form a solution or suspension.

The quantity of the first solvent is not particularly limiting provided there is enough solvent (a) to dissolve the voxelotor and form a solution, or suspend the voxelotor, and/or (b) to dissolve the fumaric acid and form a solution, or suspend the fumaric acid. The w/v ratio of voxelotor to the first solvent may be in the range of about 1 mg of voxelotor:about 1 to about 1000 μl of solvent, such as about 1 mg of voxelotor:about 1 to about 500 μl of solvent, for example about 1 mg of voxelotor:about 1 to about 150 μl of solvent, e.g. about 1 mg of voxelotor:about 1 to about 10 μl of solvent.

The voxelotor may be contacted with the first solvent at ambient temperature or less. In one embodiment, the contacting step may be carried out at one or more temperatures in the range of ≥about 0° C. to ≤about 25° C. In some embodiments, the contacting step is carried out at one or more temperatures≥about 1° C. In some embodiments, the contacting step is carried out at one or more temperatures≥about 2° C. In some embodiments, the contacting step is carried out at one or more temperatures≥about 3° C. In some embodiments, the contacting step is carried out at one or more temperatures≥about 4° C. In some embodiments, the contacting step is carried out at one or more temperatures≥about 5° C. In some embodiments, the contacting step is carried out at one or more temperatures≤about 20° C. In some embodiments, the contacting step is carried out at one or more temperatures≤about 15° C. In some embodiments, the contacting step is carried out at one or more temperatures≤about 10° C. In one embodiment, the contacting step is carried out at one or more temperatures in the range of ≥about 0° C. to ≥about 10° C., for example, about 5° C. In one embodiment, the contacting step may be carried out at about ambient temperature e.g. about 25° C.

Alternatively, the voxelotor may be contacted with the first solvent at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa). In one embodiment, the contacting step may be carried out at one or more temperatures in the range of ≥about 40° C. to about ≤60° C. In some embodiments, the contacting step is carried out at one or more temperatures≥about 41° C. In some embodiments, the contacting step is carried out at one or more temperatures≥about 42° C. In some embodiments, the contacting step is carried out at one or more temperatures≥about 43° C. In some embodiments, the contacting step is carried out at one or more temperatures≥about 44° C. In some embodiments, the contacting step is carried out at one or more temperatures≥about 45° C. In some embodiments, the contacting step is carried out at one or more temperatures≥about 46° C. In some embodiments, the contacting step is carried out at one or more temperatures≥about 47° C. In some embodiments, the contacting step is carried out at one or more temperatures≥about 48° C. In some embodiments, the contacting step is carried out at one or more temperatures≥about 49° C. In some embodiments, the contacting step is carried out at one or more temperatures≥about 50° C. In some embodiments, the contacting step is carried out at one or more temperatures≥about 59° C. In some embodiments, the contacting step is carried out at one or more temperatures≤about 58° C. In some embodiments, the contacting step is carried out at one or more temperatures≤about 57° C. In some embodiments, the contacting step is carried out at one or more temperatures≤about 56° C. In some embodiments, the contacting step is carried out at one or more temperatures≤about 55° C. In some embodiments, the contacting step is carried out at one or more temperatures≤about 54° C. In some embodiments, the contacting step is carried out at one or more temperatures≤about 53° C. In some embodiments, the contacting step is carried out at one or more temperatures≤about 52° C. In some embodiments, the contacting step is carried out at one or more temperatures≤about 51° C. In one embodiment, the contacting step is carried out at one or more temperatures in the range of ≥about 45° C. to ≤about 55° C. In one embodiment, the contacting step is carried out at a temperature of about 50° C.

The dissolution or suspension of voxelotor may be encouraged through the use of an aid such as stirring, shaking and/or sonication. Additional solvent may be added to aid the dissolution or suspension of the voxelotor.

The period of time for which the mixture of voxelotor and solvent is treated at the desired temperature is not particularly limiting. In one embodiment, the period of time may be from about 1 minute to about 24 hours, for example, about 5 minutes.

When fumaric acid is inputted into the reaction as a solid, the w/v ratio of fumaric acid to the first solvent may be in the range of about 1 mg of fumaric acid:about 1 to about 1000 μl of solvent, such as about 1 mg of fumaric acid:about 1 to about 500 μl of solvent, for example about 1 mg of fumaric acid:about 1 to about 150 μl of solvent, e.g. about 1 mg of fumaric acid:about 1 to about 20 μl of solvent.

When fumaric acid is inputted into the reaction as a solution in a solvent selected from methanol and/or TBME, the w/v ratio of fumaric acid to the solvent may be in the range of about 1 mg of fumaric acid:about 1 to about 1000 μl of solvent, such as about 1 mg of fumaric acid:about 1 to about 500 μl of solvent, for example about 1 mg of fumaric acid:about 1 to about 150 μl of solvent, e.g. about 1 mg of fumaric acid:about 1 to about 25 μl of solvent. In this instance, the solution of fumaric acid may be added to a solution/suspension of voxelotor.

When a solid admixture of voxelotor and fumaric acid is contacted with methanol and/or MTBE, the w/v ratio of the voxelotor to solvent may be in the range of about 1 mg of voxelotor:about 1 to about 1000 μl of solvent, such as about 1 mg of voxelotor:about 1 to about 500 μl of solvent, for example about 1 mg of voxelotor:about 1 to about 150 μl of solvent, e.g. about 1 mg of voxelotor:about 1 to about 10 μl of solvent. In this instance, the w/v of fumaric acid to solvent may be in the range of about 1 mg of fumaric acid:about 1 to about 1000 μl of solvent, such as about 1 mg of fumaric acid:about 1 to about 500 μl of solvent, for example about 1 mg of fumaric acid:about 1 to about 150 μl of solvent, e.g. about 1 mg of fumaric acid:about 1 to about 20 μl of solvent.

The period of time for which the mixture of voxelotor, fumaric acid and solvent is treated at the desired temperature is not particularly limiting. In one embodiment, the period of time may be from about 1 minute to about 24 hours, for example, about 1 hour.

After the combination of voxelotor, fumaric acid and solvent, the reaction mixture may be treated for a period of time at ambient temperature or less as described above in connection with first solvent.

Alternatively, the reaction mixture may be treated for a period of time at a temperature greater than ambient i.e. greater than 30° C. and below the boiling point of the reaction mixture as described above in connection with the first solvent.

The reaction mixture may be left for a further period of time, e.g. about 1 minute to about 24 hours, such as about 1 hour.

The solution or suspension may then be cooled such that the resulting solution or suspension has a temperature below that of the solution or suspension of step (a), (a2), or (a1′). The rate of cooling may be from about 0.05° C./minute to about 2° C./minute, such as about 0.1° C./minute to about 1.5° C./minute, for example about 0.1° C./minute or 0.5° C./minute. A suspension may eventually be observed on cooling a solution of the reaction mixture.

The solution or suspension may be cooled to ambient temperature or a temperature of less than ambient temperature. In one embodiment, the solution or suspension may be cooled to one or more temperatures in the range of ≥about 0° C. to about ≤20° C. In some embodiments, the solution or suspension is cooled to one or more temperatures≥about 1° C. In some embodiments, the solution or suspension is cooled to one or more temperatures≥about 2° C. In some embodiments, the solution or suspension is cooled to one or more temperatures≥about 3° C. In some embodiments, the solution or suspension is cooled to one or more temperatures≥about 4° C. In some embodiments, the solution or suspension is cooled to one or more temperatures≥about 5° C. In some embodiments, the solution or suspension is cooled to one or more temperatures≤about 15° C. In some embodiments, the solution or suspension is cooled to one or more temperatures≤about 14° C. In some embodiments, the solution or suspension is cooled to one or more temperatures≤about 13° C. In some embodiments, the solution or suspension is cooled to one or more temperatures≤about 12° C. In some embodiments, the solution or suspension may be cooled to one or more temperatures≤about 11° C. In some embodiments, the solution or suspension is cooled to one or more temperatures≤about 10° C. In one embodiment, the solution or suspension is cooled to one or more temperatures in the range of about 5° C. to about 10° C., for example, about 5° C.

The reaction mixture may be left for a further period of time, e.g. about 1 minute to about 10 days at the desired temperature. In one embodiment, the reaction mixture was left at a temperature less than ambient temperature for about 7 days.

In step (b), the voxelotor hemi fumaric acid molecular complex is recovered as a crystalline solid. The crystalline molecular complex may be recovered by directly by filtering, decanting or centrifuging. If desired, the suspension may be mobilised with additional portions of the solvent (e.g. methanol and/or TBME) prior to recovery of the crystalline solid. Alternatively, a proportion or substantially all of the solvent may be evaporated prior to recovery of the crystalline solid.

Howsoever the crystalline molecular complex is recovered, the separated molecular complex may be washed with solvent (e.g. one or more of the solvents described above) and dried.

Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline molecular complex may be left to dry under ambient temperature naturally i.e. without the active application of vacuum. It is preferred that the drying conditions are maintained below the point at which the molecular complex degrades and so when the molecular complex is known to degrade within the temperature or pressure ranges given above, the drying conditions should be maintained below the degradation temperature or vacuum.

Steps (a)→(b), (a1)→(a2)→(b), and (a1′)→(b) may be carried out one or more times (e.g. 1, 2, 3, 4 or 5 times). When steps (a)→(b), (a1)→(a2)→(b), and (a1′)→(b) are carried out more than once (e.g. 2, 3, 4 or 5 times), one or more of the steps may be optionally seeded as appropriate with crystalline voxelotor fumaric acid molecular complex (which was previously prepared and isolated by a method described herein).

The inventors envisage that the crystalline voxelotor hemi fumaric acid molecular complex described above may be prepared by a process comprising the steps of:
(a) providing an admixture of voxelotor and fumaric acid; and
(b) feeding the admixture through an extruder to form voxelotor hemi fumaric acid molecular complex.

The admixture is a blend of voxelotor and fumaric acid. The admixture may be prepared by mixing voxelotor and fumaric acid by any suitable means, e.g. by using a tubular blender, for a suitable period of time e.g. about 30 minutes. It is desirable but not essential to prepare a homogeneous blend of voxelotor and fumaric acid.

The fumaric acid may be present in stoichiometric or excess molar equivalents to the voxelotor. In one embodiment, the fumaric acid is present in stoichiometric quantities. The molar ratio of voxelotor to fumaric acid may be in the range of about 1 mole of voxelotor:about 0.3 to about 1 moles of fumaric acid, for example about 1 mole of voxelotor:about 0.4 to about 0.7 moles of fumaric acid. In one embodiment, the molar ratio of voxelotor to fumaric acid may be about 1 mole of voxelotor:about 0.5 moles of fumaric acid.

The molecular complex does not form on preparing the admixture. The voxelotor and fumaric acid co-crystallise to form the molecular complex on feeding the admixture through the extruder.

The extruder is as described above for voxelotor hemi propylene glycol solvate. A solvent may be utilised in the feeding section and/or the heating section.

The admixture may be fed into the feeding section at any suitable speed. For example, the speed of the feeding section may be from about 1 rpm to about 100 rpm. In one embodiment, the speed may be from about 5 rpm to about 80 rpm.

In certain embodiments, solvent is added to the admixture as the admixture is fed into the feeding section. Alternatively or in addition, a solvent may be added one or more times (e.g. 1, 2, 3, 4, or 5 times) in one or more zones (e.g. 1, 2, 3, 4, or 5 zones) of the heating section as the admixture traverses the heating section. This may be advantageous in preventing the admixture drying out as the material moves through the heating section.

The quantity of solvent added is not particularly limiting provided sufficient solvent is added to moisten (i.e. “wet”) the admixture but not so large a quantity that the admixture becomes too liquid.

The heating section may be heated to a single temperature across its length or it may be divided into more than one (e.g. 2, 3, 4, or 5) zones, each of which may be heated independently of the other zones. The temperature of the heating section or each zone is not particularly limiting provided that on exiting the heating section the voxelotor and fumaric acid have co-crystallised to form the molecular complex and none of voxelotor, fumaric acid and/or the molecular complex have substantially degraded or substantially decomposed.

When the extruder comprises screws, the screw (or screws) and the heating section may coincide i.e. the screw (or screws) may also be the heating section.

The speed at which the screw (or screws) rotate may be any suitable speed. For example, the speed of the screw (or screws) may be from about 1 rpm to about 500 rpm. In one embodiment, the speed may be from about 5 rpm to about 400 rpm, such as about 10 rpm to about 100 rpm.

The voxelotor hemi fumaric acid molecular complex is recovered as a crystalline solid. The crystalline molecular complex may be recovered by simply collecting the crystalline product. If desired, a proportion of the solvent (if present) may be evaporated prior to recovery of the crystalline solid.

Howsoever the crystalline molecular complex is recovered, the separated molecular complex may be dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline molecular complex may be left to dry under ambient temperature naturally i.e. without the active application of vacuum. It is preferred that the drying conditions are maintained below the point at which the molecular complex degrades and so when the molecular complex is known to degrade within the temperature or pressure ranges given above, the drying conditions should be maintained below the degradation temperature or vacuum.

In another aspect, the present invention relates to a pharmaceutical composition comprising crystalline voxelotor hemi fumaric acid molecular complex as described herein and a pharmaceutically acceptable excipient.

In another aspect, the present invention relates to a method for treating a condition associated with oxygen deficiency in a patient comprising administering a therapeutically effective amount of crystalline voxelotor hemi fumaric acid molecular complex as described herein to the patient. The condition associated with oxygen deficiency may be sickle cell disease.

In another aspect, the present invention relates to crystalline voxelotor hemi fumaric acid molecular complex as described herein for use in treating a condition associated with oxygen deficiency. The condition associated with oxygen deficiency may be sickle cell disease.

Voxelotor Hemi Succinic Acid Molecular Complex

It has been discovered that voxelotor can be prepared in a well-defined and consistently reproducible succinic acid molecular complex. Moreover, a reliable and scalable method for producing this molecular complex has been developed. The voxelotor molecular complex provided by the present invention may be useful as an active ingredient in pharmaceutical formulations. In certain embodiments, the crystalline molecular complex is purifiable. In certain embodiments and depending on time, temperature and humidity, the crystalline molecular complex is stable. In certain embodiments, the crystalline molecular complex is easy to isolate and handle. In certain embodiments, the process for preparing the crystalline molecular complex is scalable.

The crystalline molecular complex described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In another aspect, the present invention provides a crystalline molecular complex of voxelotor and succinic acid. In one embodiment, the crystalline molecular complex is voxelotor hemi succinic acid molecular complex e.g. voxelotor hemi succinic acid co-crystal.

The molar ratio of voxelotor to succinic acid may be in the range of about 1 mole of voxelotor:about 0.3 to about 1 moles of succinic acid, for example about 1 mole of voxelotor:about 0.4 to about 0.7 moles of succinic acid. In one embodiment, the molar ratio of voxelotor to succinic acid may be about 1 mole of voxelotor:about 0.5 moles of succinic acid.

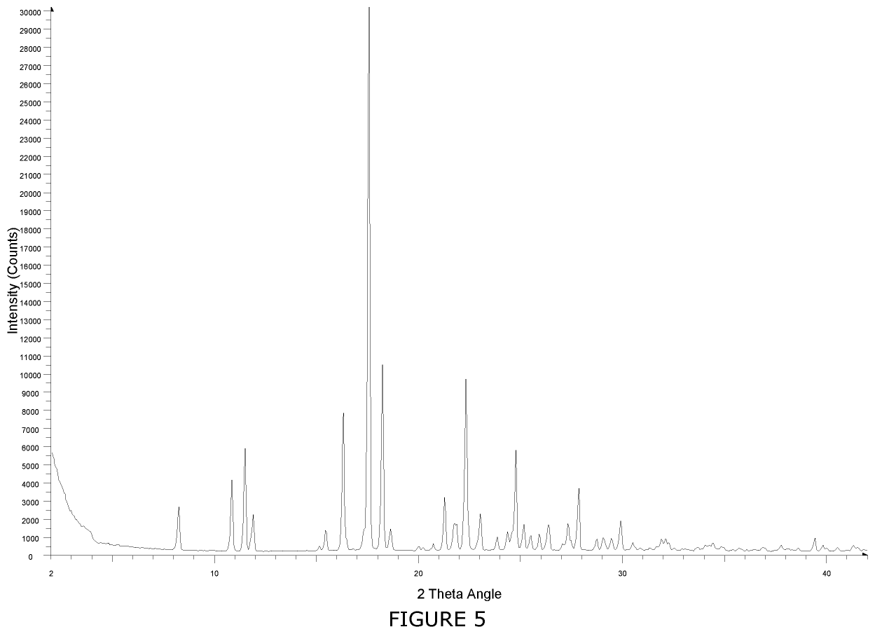

The hemi succinic acid molecular complex may have an X-ray powder diffraction pattern comprising one or more peaks (for example 1, 2, 3, 4, 5, 6, 7, 8, 9, or 10 peaks) selected from the group consisting of about 8.2, 10.8, 11.5, 11.9, 15.2, 15.5, 16.3, 17.6, 18.2, 18.6, 20.0, 20.2, 20.7, 21.3, 21.8, 22.3, 23.1, 23.9, 24.4, 24.8, 25.2, 27.4, 27.9, and 29.9 degrees two-theta±0.2 degrees two-theta. In one embodiment, the molecular complex may have the X-ray powder diffraction pattern substantially as shown in FIG. 5.

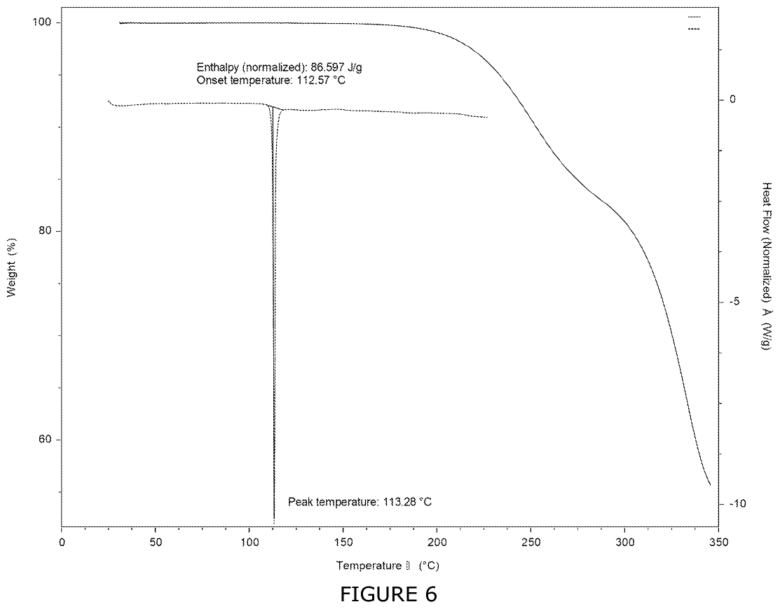

The hemi succinic acid molecular complex may have a DSC thermogram comprising an endothermic event with an onset temperature of about 112.6° C. In one embodiment, the molecular complex may have a DSC thermogram substantially as shown in FIG. 6.

The hemi succinic acid molecular complex may have a TGA thermogram comprising no substantial mass loss when heated from about ambient temperature to about 150° C. In one embodiment, the molecular complex may have a TGA thermogram substantially as shown in FIG. 6.

Thermal analysis of the hemi succinic acid molecular complex shows that there is no loss of succinic acid immediately after the melt of the solid by TGA. This shows there is a temperature window between the melt of the molecular complex (DSC event at about 112.6° C.) and sample degradation (about 170° C. by TGA), where the liquid can be cooled to reform the molecular complex. This indicates that thermal methods (such as hot melt extrusion) can be used to produce the molecular complex.

The crystalline voxelotor hemi succinic acid molecular complex formed may be free or substantially free of other polymorphic forms of voxelotor. In certain embodiments, the polymorphic purity of the molecular complex is ≥90%, ≥91%, ≥92%, ≥93%, ≥94%, ≥95% or higher. In certain embodiments, the polymorphic purity of the molecular complex is ≥95%. In certain embodiments, the polymorphic purity of the molecular complex is ≥96%. In certain embodiments, the polymorphic purity of the molecular complex is ≥97%. In certain embodiments, the polymorphic purity of the molecular complex is ≥98%. In certain embodiments, the polymorphic purity of the molecular complex is ≥99%.

The voxelotor hemi succinic acid molecular complex described above may be prepared by a process comprising reacting voxelotor and succinic acid using low energy ball milling or low energy grinding.

Succinic acid is present in sufficient quantities to form the desired molecular complex. The w/w ratio of voxelotor to succinic acid may be in the range of about 1 mg of voxelotor:about 0.1 to about 0.75 mg of succinic acid, such as about 1 mg of voxelotor:about 0.5 mg of succinic acid e.g. about 1 mg of voxelotor:about 0.2 mg of succinic acid.

When low energy ball milling is utilised, the milling process may be controlled by various parameters including the speed at which the milling takes place, the length of milling time and/or the level to which the milling container is filled.

The speed at which the milling takes place may be from about 50 rpm to about 1000 rpm. In one embodiment, the speed may be from about 75 rpm to about 750 rpm. In another embodiment, the speed may be from about 80 rpm to about 650 rpm. In one embodiment, the speed may be about 500 rpm.

Low energy grinding involves shaking the materials within a grinding container. The grinding occurs via the impact and friction of the materials within the container. The process may be controlled by various parameters including the frequency at which the grinding takes place, the length of grinding time and/or the level to which the container is filled.

The frequency at which the grinding takes place may be from about 1 Hz to about 100 Hz. In one embodiment, the frequency may be from about 10 Hz to about 70 Hz. In another embodiment, the frequency may be from about 20 Hz to about 50 Hz. In one embodiment, the frequency may be about 30 Hz.

Regardless of whether milling or grinding is used, milling or grinding media may be used to assist the reaction. In this instance, the incorporation of hard, non-contaminating media can additionally assist in the breakdown of particles where agglomeration has occurred, for example, as a result of the manufacturing process or during transit. Such breakdown of the agglomerates further enhances the reaction of voxelotor with succinic acid. The use of milling/grinding media is well-known within the field of powder processing and materials such as stabilised zirconia and other ceramics are suitable provided they are sufficiently hard or ball bearings e.g. stainless steel ball bearings.

Regardless of whether milling or grinding is used, an improvement in the process can be made by controlling the particle ratio, the size of the milling/grinding media and other parameters as are familiar to the skilled person.

The length of milling or grinding time may be from about 1 minute to about 2 days, for example, about 2 minutes to about 5 hours, such as about 20 minutes to 3 hours, e.g. about 2 hours. The length of the milling or grinding time may be a continuous or aggregate period of time. “Continuous” and “aggregate” are defined below.

The voxelotor and succinic acid may be contacted at ambient temperature or less. Alternatively, the voxelotor may be contacted with the succinic acid at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa).

The process may be carried out in the presence of a solvent such as methanol. The solvent may act to minimise particle welding. The addition of the solvent may be particularly helpful if the voxelotor and/or succinic acid being reacted has agglomerated prior to use, in which case the solvent can assist with breaking down the agglomerates.

The quantity of solvent is not particularly limiting provided there is enough solvent to dissolve, suspend or moisten the voxelotor and/or succinic acid. The w/v ratio of voxelotor to solvent may be in the range from about 1 mg of voxelotor:about 0.01 to about 1.5 μl solvent, such as about 1 mg of voxelotor:about 0.05 to about 1.0 μl solvent, for example about 1 mg of voxelotor:about 0.1 to about 0.75 μl solvent, e.g. about 1 mg of voxelotor:about 0.5 μl solvent. The solvent may be added in one portion or more than portion (e.g. 2, 3, 4, or 5 portions).

The voxelotor and succinic acid may be contacted with the solvent at ambient temperature or less. Alternatively, the voxelotor may be contacted with the solvent at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa).

When the milling or grinding time is applied for an aggregate period of time, the presence or absence of solvent may be changed for each period of time. For example, the process may comprise a first period of time in which the environment is dry (i.e. voxelotor and succinic acid are reacted together optionally with milling media in the absence of solvent), and a second period of time in which the environment is moistened (i.e. “wet”) after the addition of solvent.

The voxelotor hemi succinic acid molecular complex is recovered as a crystalline solid. The crystalline molecular complex may be recovered by directly by filtering, decanting or centrifuging. If desired, a proportion of the solvent may be evaporated prior to recovery of the crystalline solid.

Alternatively, the voxelotor hemi succinic acid molecular complex described above may be prepared by a process comprising the step of applying dual asymmetric centrifugal forces to a mixture of voxelotor and succinic acid to form the solvate.

Succinic acid is present in sufficient quantities to form the desired molecular complex. The molar ratio of voxelotor to succinic acid may be in the range of about 1 mole of voxelotor:about 0.3 to about 1 moles of succinic acid, for example about 1 mole of voxelotor:about 0.4 to about 0.7 moles of succinic acid. In one embodiment, the molar ratio of voxelotor to succinic acid may be about 1 mole of voxelotor:about 0.5 moles of succinic acid.

The voxelotor hemi succinic acid molecular complex is formed using dual asymmetric centrifugal forces. By “dual asymmetric centrifugal forces” we mean that two centrifugal forces, at an angle to each other, are simultaneously applied to the particles. In order to create an efficient mixing environment, the centrifugal forces preferably rotate in opposite directions. The Speedmixer™ by Hauschild (http://www.speedmixer.co.uk/index.php) utilises this dual rotation method whereby the motor of the Speedmixer™ rotates the base plate of the mixing unit in a clockwise direction (see FIG. 7A) and the basket is spun in an anti-clockwise direction (see FIGS. 7B and 7C).

The process may be controlled by various parameters including the rotation speed at which the process takes place, the length of processing time, the level to which the mixing container is filled, the use of milling media and/or the control of the temperature of the components within the milling pot.

The dual asymmetric centrifugal forces may be applied for a continuous period of time. By “continuous” we mean a period of time without interruption. The period of time may be from about 1 second to about 10 minutes, such as about 5 seconds to about 5 minutes, for example, about 10 seconds to about 200 seconds e.g. 2 minutes.

Alternatively, the dual asymmetric centrifugal forces may be applied for an aggregate period of time. By “aggregate” we mean the sum or total of more than one periods of time (e.g. 2, 3, 4, 5 or more times). The advantage of applying the centrifugal forces in a stepwise manner is that excessive heating of the particles can be avoided. The dual asymmetric centrifugal forces may be applied for an aggregate period of about 1 second to about 20 minutes, for example about 30 seconds to about 15 minutes and such as about 10 seconds to about 10 minutes e.g. 6 minutes. In one embodiment, the dual asymmetric centrifugal forces are applied in a stepwise manner with periods of cooling therebetween. In another embodiment, the dual asymmetric centrifugal forces may be applied in a stepwise manner at one or more different speeds.

The speed of the dual asymmetric centrifugal forces may be from about 200 rpm to about 4000 rpm. In one embodiment, the speed may be from about 300 rpm to about 3750 rpm, for example about 500 rpm to about 3500 rpm. In one embodiment, the speed may be about 3500 rpm. In another embodiment, the speed may be about 2300 rpm.

The level to which the mixing container is filled is determined by various factors which will be apparent to the skilled person. These factors include the apparent density of the voxelotor and succinic acid, the volume of the mixing container and the weight restrictions imposed on the mixer itself.

Milling media as described above may be used to assist the reaction. In certain embodiments, the dual asymmetric centrifugal forces may be applied in a stepwise manner in which milling media may be used for some, but not all, periods of time.

The process may be carried out in the presence of a solvent such as methanol or TBME. The solvent may act to minimise particle welding. The addition of the solvent may be particularly helpful if the voxelotor and/or succinic acid being reacted has agglomerated prior to use, in which case the solvent can assist with breaking down the agglomerates.

When the dual asymmetric centrifugal forces are applied for an aggregate period of time, the presence or absence of solvent may be changed for each period of time. For example, the process may comprise a first period of time in which the environment is dry (i.e. voxelotor and succinic acid are reacted together optionally with milling media in the absence of solvent), and a second period of time in which the environment is moistened (i.e. “wet”) after the addition of solvent.

The voxelotor hemi succinic acid molecular complex is recovered as a crystalline solid. The crystalline molecular complex may be recovered by directly by filtering, decanting or centrifuging. If desired, a proportion of the solvent (if present) may be evaporated prior to recovery of the crystalline solid.

Howsoever the crystalline molecular complex is recovered, the separated molecular complex may be dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline molecular complex may be left to dry under ambient temperature naturally i.e. without the active application of vacuum. It is preferred that the drying conditions are maintained below the point at which the molecular complex degrades and so when the molecular complex is known to degrade within the temperature or pressure ranges given above, the drying conditions should be maintained below the degradation temperature or vacuum.

The crystalline voxelotor hemi succinic acid molecular complex described above may be prepared by a process comprising the steps of:
(a) contacting voxelotor and succinic acid with a solvent which is tert-butyl methyl ether (TMBE); and
(b) recovering voxelotor hemi succinic acid molecular complex as a crystalline solid.

Succinic acid may be utilised as a solid, or as a solution in a solvent (e.g. methanol and/or TBME).

In one embodiment, step (a) may comprise the steps of:
(a1) contacting voxelotor with a solvent which is tert-butyl methyl ether (TMBE); and
(a2) adding succinic acid to the solution or suspension of voxelotor.

In another embodiment, step (a) may comprise the step of:
(a1′) contacting a solid admixture of voxelotor and succinic acid with a solvent which is tert-butyl methyl ether (TMBE) to form a solution or suspension.

The quantity of the TBME solvent is not particularly limiting provided there is enough solvent (a) to dissolve the voxelotor and form a solution, or suspend the voxelotor, and/or (b) to dissolve the succinic acid and form a solution, or suspend the succinic acid. The w/v ratio of voxelotor to TBME may be in the range of about 1 mg of voxelotor:about 1 to about 1000 μl of TBME, such as about 1 mg of voxelotor:about 1 to about 500 μl of TBME, for example about 1 mg of voxelotor:about 1 to about 150 μl of TBME, e.g. about 1 mg of voxelotor:about 1 to about 10 μl of TBME. In one embodiment, the w/v ratio of voxelotor to TBME may be about 1 mg of voxelotor:about 5 μl of TBME.

The voxelotor may be contacted with the TBME at ambient temperature or less. In one embodiment, the contacting step may be carried out at one or more temperatures in the range of ≥about 0° C. to about ≤25° C. In some embodiments, the contacting step is carried out at one or more temperatures≥about 1° C. In some embodiments, the contacting step is carried out at one or more temperatures≥about 2° C. In some embodiments, the contacting step is carried out at one or more temperatures≥about 3° C. In some embodiments, the contacting step is carried out at one or more temperatures≥about 4° C. In some embodiments, the contacting step is carried out at one or more temperatures≥about 5° C. In some embodiments, the contacting step is carried out at one or more temperatures≤about 20° C. In some embodiments, the contacting step is carried out at one or more temperatures≤about 15° C. In some embodiments, the contacting step is carried out at one or more temperatures≤about 10° C. In one embodiment, the contacting step is carried out at one or more temperatures in the range of ≥about 0° C. to ≥about 10° C., for example, about 5° C. In one embodiment, the contacting step may be carried out at about ambient temperature e.g. about 25° C.

Alternatively, the voxelotor may be contacted with the TBME at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa). In one embodiment, the contacting step may be carried out at one or more temperatures in the range of ≥about 40° C. to about ≤60° C. In some embodiments, the contacting step is carried out at one or more temperatures≥about 41° C. In some embodiments, the contacting step is carried out at one or more temperatures≥about 42° C. In some embodiments, the contacting step is carried out at one or more temperatures≥about 43° C. In some embodiments, the contacting step is carried out at one or more temperatures≥about 44° C. In some embodiments, the contacting step is carried out at one or more temperatures≥about 45° C. In some embodiments, the contacting step is carried out at one or more temperatures≥about 46° C. In some embodiments, the contacting step is carried out at one or more temperatures≥about 47° C. In some embodiments, the contacting step is carried out at one or more temperatures≥about 48° C. In some embodiments, the contacting step is carried out at one or more temperatures≥about 49° C. In some embodiments, the contacting step is carried out at one or more temperatures≥about 50° C. In some embodiments, the contacting step is carried out at one or more temperatures≤about 59° C. In some embodiments, the contacting step is carried out at one or more temperatures≤about 58° C. In some embodiments, the contacting step is carried out at one or more temperatures≤about 57° C. In some embodiments, the contacting step is carried out at one or more temperatures≤about 56° C. In some embodiments, the contacting step is carried out at one or more temperatures≥about 55° C. In some embodiments, the contacting step is carried out at one or more temperatures≤about 54° C. In some embodiments, the contacting step is carried out at one or more temperatures≤about 53° C. In some embodiments, the contacting step is carried out at one or more temperatures≤about 52° C. In some embodiments, the contacting step is carried out at one or more temperatures≤about 51° C. In one embodiment, the contacting step is carried out at one or more temperatures in the range of ≥about 45° C. to ≤about 55° C. In one embodiment, the contacting step is carried out at a temperature of about 50° C.

The dissolution or suspension of voxelotor may be encouraged through the use of an aid such as stirring, shaking and/or sonication. Additional solvent may be added to aid the dissolution or suspension of the voxelotor.

The period of time for which the mixture of voxelotor and TBME is treated at the desired temperature is not particularly limiting. In one embodiment, the period of time may be from about 1 minute to about 24 hours, for example, about 2 hours.

When succinic acid is inputted into the reaction as a solid, the w/v ratio of succinic acid to TBME may be in the range of about 1 mg of succinic acid:about 1 to about 1000 μl of TBME, such as about 1 mg of succinic acid:about 1 to about 500 μl of TBME, for example about 1 mg of succinic acid:about 1 to about 150 μl of TBME, e.g. about 1 mg of succinic acid:about 1 to about 35 μl of TBME. In one embodiment, the w/v ratio of succinic acid to TBME may be about 1 mg succinic acid:about 29 μl of solvent.

Succinic acid may be inputted into the reaction as a solution in methanol and/or TBME. In this instance, the w/v ratio of succinic acid to TBME may be in the range of about 1 mg of succinic acid:about 1 to about 1000 μl of TBME, such as about 1 mg of succinic acid:about 1 to about 500 μl of TBME, for example about 1 mg of succinic acid:about 1 to about 150 μl of TBME, e.g. about 1 mg of succinic acid:about 1 to about 25 μl of TBME. In this instance, the solution of succinic acid may be added to a solution/suspension of voxelotor.

When a solid admixture of voxelotor and succinic acid is contacted with MTBE, the w/v ratio of voxelotor to TBME may be in the range of about 1 mg of voxelotor:about 1 to about 1000 μl of TBME, such as about 1 mg of voxelotor:about 1 to about 500 μl of TBME, for example about 1 mg of voxelotor:about 1 to about 150 μl of TBME, e.g. about 1 mg of voxelotor:about 1 to about 10 μl of TBME. In one embodiment, the w/v ratio of voxelotor to TBME may be about 1 mg of voxelotor:about 5 μl of TBME. In this instance, the w/v ratio of succinic acid to TBME may be in the range of about 1 mg of succinic acid:about 1 to about 1000 μl of TBME, such as about 1 mg of succinic acid:about 1 to about 500 μl of TBME, for example about 1 mg of succinic acid:about 1 to about 150 μl of TBME, e.g. about 1 mg of succinic acid:about 1 to about 35 μl of TBME. In one embodiment, the w/v ratio of succinic acid to TBME may be about 1 mg succinic acid:about 29 μl of solvent.

The period of time for which the mixture of voxelotor, succinic acid and solvent is treated at the desired temperature is not particularly limiting. In one embodiment, the period of time may be from about 1 minute to about 24 hours, for example, about 1 hour.

After the combination of voxelotor, succinic acid and solvent, the reaction mixture may be treated for a period of time at ambient temperature or less as described above in connection with first solvent.

Alternatively, the reaction mixture may be treated for a period of time at one or more temperatures greater than ambient i.e. greater than 30° C. and below the boiling point of the reaction mixture as described above in connection with the first solvent.

The reaction mixture may be left for a further period of time, e.g. about 1 minute to about 24 hours, such as about 2 hours.

The solution or suspension may then be cooled such that the resulting solution or suspension has a temperature below that of the solution or suspension of step (a), (a2), or (a1′). The rate of cooling may be from about 0.05° C./minute to about 2° C./minute, such as about 0.1° C./minute to about 1.5° C./minute, for example about 0.1° C./minute or 0.5° C./minute. A suspension may eventually be observed on cooling a solution of the reaction mixture.

The solution or suspension may be cooled to ambient temperature or a temperature of less than ambient temperature. In one embodiment, the solution or suspension may be cooled to one or more temperatures in the range of ≥about 0° C. to about ≤20° C. In some embodiments, the solution or suspension is cooled to one or more temperatures≥about 1° C. In some embodiments, the solution or suspension is cooled to one or more temperatures≥about 2° C. In some embodiments, the solution or suspension is cooled to one or more temperatures≥about 3° C. In some embodiments, the solution or suspension is cooled to one or more temperatures≥about 4° C. In some embodiments, the solution or suspension is cooled to one or more temperatures≥about 5° C. In some embodiments, the solution or suspension is cooled to one or more temperatures≥about 15° C. In some embodiments, the solution or suspension is cooled to one or more temperatures≥about 14° C. In some embodiments, the solution or suspension is cooled to one or more temperatures≥about 13° C. In some embodiments, the solution or suspension is cooled to one or more temperatures≥about 12° C. In some embodiments, the solution or suspension may be cooled to one or more temperatures≥about 11° C. In some embodiments, the solution or suspension is cooled to one or more temperatures≥about 10° C. In one embodiment, the solution or suspension is cooled to one or more temperatures in the range of about 5° C. to about 10° C., for example, about 5° C.

The reaction mixture may be left for a further period of time, e.g. about 1 minute to about 10 days at the desired temperature.

In step (b), the voxelotor hemi succinic acid molecular complex is recovered as a crystalline solid. The crystalline molecular complex may be recovered by directly by filtering, decanting or centrifuging. If desired, the suspension may be mobilised with additional portions of the solvent (e.g. TBME) prior to recovery of the crystalline solid. Alternatively, a proportion or substantially all of the solvent may be evaporated prior to recovery of the crystalline solid.

Howsoever the crystalline molecular complex is recovered, the separated molecular complex may be washed with solvent (e.g. TBME) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline molecular complex may be left to dry under ambient temperature naturally i.e. without the active application of vacuum. It is preferred that the drying conditions are maintained below the point at which the molecular complex degrades and so when the molecular complex is known to degrade within the temperature or pressure ranges given above, the drying conditions should be maintained below the degradation temperature or vacuum.

Steps (a)→(b), (a1)→(a2)→(b), and (a1′)→(b) may be carried out one or more times (e.g. 1, 2, 3, 4 or 5 times). When steps (a)→(b), (a1)→(a2)→(b), and (a1′)→(b) are carried out more than once (e.g. 2, 3, 4 or 5 times), one or more of the steps may be optionally seeded as appropriate with crystalline voxelotor succinic acid molecular complex (which was previously prepared and isolated by a method described herein).

The inventors envisage that the crystalline voxelotor hemi succinic acid molecular complex described above may be prepared by a process comprising the steps of:
(a) providing an admixture of voxelotor and succinic acid; and
(b) feeding the admixture through an extruder to form voxelotor hemi succinic acid molecular complex.

The admixture is a blend of voxelotor and succinic acid. The admixture may be prepared by mixing voxelotor and succinic acid by any suitable means, e.g. by using a tubular blender, for a suitable period of time e.g. about 30 minutes. It is desirable but not essential to prepare a homogeneous blend of voxelotor and succinic acid.

The succinic acid may be present in stoichiometric or excess molar equivalents to the voxelotor. In one embodiment, the succinic acid is present in stoichiometric quantities. The molar ratio of voxelotor to succinic acid may be in the range of about 1 mole of voxelotor:about 0.3 to about 1 moles of succinic acid, for example about 1 mole of voxelotor:about 0.4 to about 0.7 moles of succinic acid. In one embodiment, the molar ratio of voxelotor to succinic acid may be about 1 mole of voxelotor:about 0.5 moles of succinic acid.

The molecular complex does not form on preparing the admixture. The voxelotor and fumaric acid co-crystallise to form the molecular complex on feeding the admixture through the extruder.

The extruder is as described above for voxelotor hemi propylene glycol solvate. A solvent may be utilised in the feeding section and/or heating section.

The admixture may be fed into the feeding section at any suitable speed. For example, the speed of the feeding section may be from about 1 rpm to about 100 rpm. In one embodiment, the speed may be from about 5 rpm to about 80 rpm.

In certain embodiments, solvent is added to the admixture as the admixture is fed into the feeding section. Alternatively or in addition, a solvent may be added one or more times (e.g. 1, 2, 3, 4, or 5 times) in one or more zones (e.g. 1, 2, 3, 4, or 5 zones) of the heating section as the admixture traverses the heating section. This may be advantageous in preventing the admixture drying out as the material moves through the heating section.

The quantity of solvent added is not particularly limiting provided sufficient solvent is added to moisten (i.e. “wet”) the admixture but not so large a quantity that the admixture becomes too liquid. When the extruder is a twin screw extruder, the w/v ratio of total solids (voxelotor and succinic acid) to total solvent added may be in the range of about 1 g total solids:about 0.1 to about 2 ml of total solvent added, such as about 1 g total solids:about 0.5 ml to about 1.5 ml of total solvent, e.g. about 1 g total solids:about 0.75 ml to about 1.25 ml of total solvent. In one embodiment, the w/v ratio of total solids (voxelotor and succinic acid) to total solvent is about 1 g total solids:about 1 ml of total solvent.

The heating section may be heated to a single temperature across its length or it may be divided into more than one (e.g. 2, 3, 4, or 5) zones, each of which may be heated independently of the other zones. The temperature of the heating section or each zone is not particularly limiting provided that on exiting the heating section the voxelotor and succinic acid have co-crystallised to form the molecular complex and none of voxelotor, succinic acid and/or the molecular complex have substantially degraded or substantially decomposed.

When the extruder comprises screws, the screw (or screws) and the heating section may coincide i.e. the screw (or screws) may also be the heating section.

The speed at which the screw (or screws) rotate may be any suitable speed. For example, the speed of the screw (or screws) may be from about 1 rpm to about 500 rpm. In one embodiment, the speed may be from about 5 rpm to about 400 rpm, such as about 10 rpm to about 100 rpm.

The voxelotor hemi succinic acid molecular complex is recovered as a crystalline solid. The crystalline molecular complex may be recovered by simply collecting the crystalline product. If desired, a proportion of the solvent (if present) may be evaporated prior to recovery of the crystalline solid.

Howsoever the crystalline molecular complex is recovered, the separated molecular complex may be dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline molecular complex may be left to dry under ambient temperature naturally i.e. without the active application of vacuum. It is preferred that the drying conditions are maintained below the point at which the molecular complex degrades and so when the molecular complex is known to degrade within the temperature or pressure ranges given above, the drying conditions should be maintained below the degradation temperature or vacuum.

In another aspect, the present invention relates to a pharmaceutical composition comprising crystalline voxelotor hemi succinic acid molecular complex as described herein and a pharmaceutically acceptable excipient.

In another aspect, the present invention relates to a method for treating a condition associated with oxygen deficiency in a patient comprising administering a therapeutically effective amount of crystalline voxelotor hemi succinic acid molecular complex as described herein to the patient. The condition associated with oxygen deficiency may be sickle cell disease.

In another aspect, the present invention relates to crystalline voxelotor hemi succinic acid molecular complex as described herein for use in treating a condition associated with oxygen deficiency. The condition associated with oxygen deficiency may be sickle cell disease.

Embodiments and/or optional features of the invention have been described above. Any aspect of the invention may be combined with any other aspect of the invention, unless the context demands otherwise. Any of the embodiments or optional features of any aspect may be combined, singly or in combination, with any aspect of the invention, unless the context demands otherwise.

The invention will now be described further by reference to the following examples, which are intended to illustrate but not limit, the scope of the invention.

### EXAMPLES

1 Instrument and Methodology Details

1.1 X-Ray Powder Diffraction (XRPD)

XRPD diffractograms were collected on a Bruker D8 diffractometer using Cu Kα radiation (40 kV, 40 mA) and a θ-2θ goniometer fitted with a Ge monochromator. The incident beam passes through a 2.0 mm divergence slit followed by a 0.2 mm anti-scatter slit and knife edge. The diffracted beam passes through an 8.0 mm receiving slit with 2.5° Soller slits followed by the Lynxeye Detector. The software used for data collection and analysis was Diffrac Plus XRD Commander and Diffrac Plus EVA respectively.

Samples were run under ambient conditions as flat plate specimens using powder as received. The sample was prepared on a polished, zero-background (510) silicon wafer by gently pressing onto the flat surface or packed into a cut cavity. The sample was rotated in its own plane.

The details of the standard data collection method are:
Angular range: 2 to 42° 2θ
Step size: 0.05° 2θ
Collection time: 0.5 s/step (total collection time: 6.40 min)

1.2 Differential Scanning Calorimetry (DSC)

DSC data were collected on a TA Instruments Q2000 equipped with a 50 position auto-sampler. Typically, 0.5-3 mg of each sample, in a pin-holed aluminium pan, was heated at 10° C./min from 25° C. to 250° C. A purge of dry nitrogen at 50 ml/min was maintained over the sample.

Modulated temperature DSC was carried out using an underlying heating rate of 2° C./min and temperature modulation parameters of ±0.636° C. (amplitude) every 60 seconds (period).

The instrument control software was Advantage for Q Series and Thermal Advantage and the data were analysed using Universal Analysis or TRIOS.

1.3 Thermo-Gravimetric Analysis (TGA)

1.3.1 TA Instruments Q500

TGA data were collected on a TA Instruments Q500 TGA, equipped with a 16 position auto-sampler. Typically, 5-10 mg of each sample was loaded onto a pre-tared aluminium DSC pan and heated at 10° C./min from ambient temperature to 350° C. A nitrogen purge at 60 ml/min was maintained over the sample.

The instrument control software was Advantage for Q Series and Thermal Advantage and the data were analysed using Universal Analysis or TRIOS.

1.3.2 TA Instruments Discovery TGA

TGA data were collected on a TA Instruments Discovery TGA, equipped with a 25 position auto-sampler. Typically, 5-10 mg of each sample was loaded onto a pre-tared aluminium DSC pan and heated at 10° C./min from ambient temperature to 350° C. A nitrogen purge at 25 ml/min was maintained over the sample.

The instrument control software was TRIOS and the data were analysed using TRIOS or Universal Analysis.

### Voxelotor Hemi Propylene Glycol Solvate

### Example 1

Voxelotor (29 mg) was weighed into a HPLC vial. The solid was wetted with propylene glycol (15 μl) and two 3 mm stainless steel grinding balls were added to the vial. The sample was ground for 2 hours at 500 rpm in a planetary mill. After grinding the vial was left uncapped overnight to dry.

### Example 2

Voxelotor (2.00 g) was dissolved in TBME (8.00 ml, 4 vol) at 50° C. Propylene glycol (650 μl, 1.5 eq) was added to the solution before cooling to 5° C. at 0.1° C./min. The resulting suspension was filtered and dried under suction.

### Example 3

Voxelotor (29 mg) was dissolved in isopropyl acetate (150 μl, 5 vol) at 50° C. Propylene glycol (0.5 eq, 12 μl) was added to the resulting solution which was cooled to 5° C. at 0.1° C./min. The resulting suspension was filtered and dried under suction.

### Example 4

Voxelotor (29 mg) was dissolved in diethyl ether (150 μl, 5 vol) at 50° C. Propylene glycol (0.5 eq, 12 μl) was added to the resulting solution which was cooled to 5° C. at 0.1° C./min. The resulting suspension was filtered and dried under suction.

### Example 5

Voxelotor (29 mg) was dissolved in 2-methyl THF (150 μl, 5 vol) at 50° C. Propylene glycol (0.5 eq, 12 μl) was added to the resulting solution which was cooled to 5° C. at 0.1° C./min. The resulting suspension was filtered and dried under suction.

### Example 6

Voxelotor (5.0 g) was dissolved TBME (20.0 ml, 4 vol) and heated to 50° C. Propylene glycol (0.6 eq, 650 μl) was added to the resulting solution which was cooled to 45° C. and seeded with voxelotor hemi propylene glycol solvate (Example 2) before cooling to 5° C. at 0.5° C./min. At 15° C., heptane (20 ml) was added to the suspension. After cooling to 5° C., the resulting suspension was filtered and dried under suction. The isolated solid was dried under vacuum at RT for 1 hr.

Characterisation of Voxelotor Hemi Propylene Glycol Solvate

FIG. 1 shows a representative XRPD pattern of voxelotor hemi propylene glycol solvate. The following table provides an XRPD peak list for the solvate:

| Angle | Intensity |
|---|---|
| (2-Theta °) | (%) |
| 8.6 | 24.8 |
| 8.8 | 29.0 |
| 11.3 | 15.8 |
| 12.6 | 5.5 |
| 12.9 | 8.4 |
| 14.5 | 8.0 |
| 15.0 | 31.8 |
| 15.5 | 16.0 |
| 15.6 | 28.8 |
| 16.0 | 3.7 |
| 16.8 | 4.2 |
| 17.1 | 22.6 |
| 17.7 | 14.9 |
| 18.0 | 1.6 |
| 18.6 | 7.4 |
| 19.1 | 3.3 |
| 19.7 | 2.3 |
| 20.2 | 5.1 |
| 20.9 | 7.5 |
| 22.8 | 13.1 |
| 23.1 | 12.7 |
| 23.7 | 100.0 |
| 24.2 | 43.6 |
| 25.1 | 3.9 |
| 25.4 | 7.8 |
| 25.9 | 48.3 |
| 26.7 | 11.7 |
| 27.2 | 5.6 |
| 28.8 | 5.7 |
| 30.3 | 6.6 |
| 31.6 | 8.4 |
| 32.4 | 7.5 |

Voxelotor hemi propylene glycol solvate was also characterised by TGA and DSC analysis (see FIG. 2).

### Voxelotor Hemi Fumaric Acid Molecular Complex

### Example 7

Voxelotor (300 mg) was dissolved in methanol (1.5 ml, 5 vol) at 50° C. A portion of the warm voxelotor solution (250 μl, ˜50 mg) was added to a vial containing solid fumaric acid (18 mg, 1 eq) and was left to stir at 50° C. for 1 hour before cooling to 5° C. at 0.1° C./min. After cooling, the resulting suspension was kept at 5° C. for 7 days before filtration and drying under suction.

### Example 8

A solid mixture of Voxelotor (1.00 g) and fumaric acid (173 mg, 0.5 eq) was dissolved in methanol (2.5 ml, 2.5 vol) at 50° C. The resulting solution was stirred at 50° C. for 1 hour before cooling to 5° C. at 0.1° C./min. The resulting thick suspension was transferred onto filter paper to dry ambiently.

### Example 9

Voxelotor (1.00 g) was dissolved in TBME (4.00 ml, 4 vol) at 50° C. Fumaric acid (0.6 eq, 210 mg in 4 ml methanol) was added to the solution which was cooled to 20° C. and then seeded with voxelotor hemi fumaric acid molecular complex (Example 8). The sample was further cooled to 5° C. at 0.1° C./min. The resulting suspension was filtered and dried under suction.

### Example 10

A solid mixture of Voxelotor (5.00 g) and fumaric acid (0.6 eq, 1035 mg) were dissolved methanol (12.5 ml, 2.5 vol) and heated to 50° C. The resulting solution was cooled to 45° C. and then seeded with voxelotor hemi fumaric acid molecular complex (Example 8) before cooling to 5° C. at 0.5° C./min. At 5° C., the resulting thick suspension was treated with TBME (5 ml) to mobilise the solid. After a further 2 hrs at 5° C., the suspension was filtered and dried under suction. The isolated solid was dried under vacuum at RT for 1 hr.

Characterisation of Voxelotor Hemi Fumaric Acid Molecular Complex

FIG. 3 shows a representative XRPD pattern of voxelotor hemi fumaric acid molecular complex. The following table provides an XRPD peak list for the molecular complex:

| Angle | Intensity |
|---|---|
| (2-Theta °) | (%) |
| 5.3 | 54.5 |
| 6.9 | 25.5 |
| 11.2 | 90.4 |
| 12.5 | 27.9 |
| 12.8 | 11.4 |
| 13.4 | 2.6 |
| 13.9 | 8.5 |
| 14.2 | 7.7 |
| 15.1 | 7.1 |
| 15.9 | 100.0 |
| 16.2 | 4.0 |
| 17.3 | 3.5 |
| 17.5 | 4.7 |
| 17.8 | 10.1 |
| 18.7 | 9.4 |
| 19.4 | 2.9 |
| 19.6 | 3.0 |
| 20.3 | 3.6 |
| 20.9 | 11.5 |
| 21.2 | 20.2 |
| 21.7 | 4.6 |
| 22.3 | 6.8 |
| 22.6 | 31.0 |
| 23.1 | 8.4 |
| 23.3 | 5.5 |
| 24.1 | 3.0 |
| 24.4 | 6.0 |
| 24.8 | 3.3 |
| 25.1 | 6.2 |
| 25.8 | 4.9 |
| 25.9 | 5.2 |
| 26.4 | 27.6 |
| 27.7 | 5.8 |

Voxelotor hemi fumaric acid molecular complex was also characterised by TGA and DSC analysis (see FIG. 4).

### Voxelotor Hemi Succinic Acid Molecular Complex

### Example 11

Voxelotor (30 mg) and 0.5 eq succinic acid (6.1 mg) were weighed into a HPLC vial. The solids were wetted with methanol (15 μl, 0.5 vol) and two 3 mm stainless steel grinding balls were added to the vial. The sample was ground for 2 hours at 500 rpm in planetary mill. After grinding, the vial was left uncapped to dry the solid before analysis by XRPD.

### Example 12

Voxelotor (1.00 g) and 0.5 eq succinic acid (175 mg) were weighed into a 10 ml stainless steel grinding jar with a 7 mm stainless steel grinding ball. The solid mixture was ground at 30 Hz for 2 mins in Retch mill to homogenise the solid before being wetted with methanol (500 μl, 0.5 vol). The sample was further ground for 30 min at 30 Hz four times (total time 120 mins).

### Example 13

A solid mixture of Voxelotor (5.00 g) and succinic acid (0.5 eq, 875 mg) were suspended in TBME (25.0 ml, 5 vol) and heated to 50° C. The resulting suspension was stirred at 50° C. for 2 hrs after which the suspension was then cooled to 5° C. at 0.5° C./min. At 5° C., the suspension was filtered and dried under suction.

Characterisation of Voxelotor Hemi Succinic Acid Molecular Complex

FIG. 5 shows a representative XRPD pattern of voxelotor hemi succinic acid molecular complex. The following table provides an XRPD peak list for the molecular complex:

| Angle | |
|---|---|
| (2- | Intensity |
| Theta °) | (%) |
| 8.2 | 8.6 |
| 10.8 | 13.6 |
| 11.5 | 19.3 |
| 11.9 | 7.3 |
| 15.2 | 1.5 |
| 15.5 | 4.4 |
| 16.3 | 25.8 |
| 17.6 | 100.0 |
| 18.2 | 34.6 |
| 18.6 | 4.6 |
| 20.0 | 1.4 |
| 20.2 | 1.2 |
| 20.7 | 1.9 |
| 21.3 | 10.4 |
| 21.8 | 5.7 |
| 22.3 | 32.0 |
| 23.1 | 7.4 |
| 23.9 | 3.1 |
| 24.4 | 4.1 |
| 24.8 | 19.0 |
| 25.2 | 5.5 |
| 27.4 | 5.6 |
| 27.9 | 12.1 |
| 29.9 | 6.1 |

Voxelotor hemi succinic acid molecular complex was also characterised by TGA and DSC analysis (see FIG. 6).

1. A crystalline form of voxelotor which is crystalline voxelotor hemi succinic acid molecular complex.

2. A crystalline form of voxelotor according to claim 1, wherein the crystalline voxelotor hemi succinic acid molecular complex has an X-ray powder diffraction pattern comprising one or more peaks selected from the group consisting of about 8.2, 10.8, 11.5, 11.9, 15.2, 15.5, 16.3, 17.6, 18.2, 18.6, 20.0, 20.2, 20.7, 21.3, 21.8, 22.3, 23.1, 23.9, 24.4, 24.8, 25.2, 27.4, 27.9, and 29.9 degrees two-theta±0.2 degrees two-theta.

3. A crystalline form of voxelotor according to claim 2, which has X-ray powder diffraction pattern substantially as shown in FIG. 5.

4. A crystalline form of voxelotor which is crystalline voxelotor hemi fumaric acid molecular complex.

5. A crystalline form of voxelotor according to claim 4, wherein the crystalline voxelotor hemi fumaric acid molecular complex has an X-ray powder diffraction pattern comprising one or more peaks selected from the group consisting of about 5.3, 6.9, 11.2, 12.5, 12.8, 13.4, 13.9, 14.2, 15.1, 15.9, 16.2, 17.3, 17.5, 17.8, 18.7, 19.4, 19.6, 20.3, 20.9, 21.2, 21.7, 22.3, 22.6, 23.1, 23.3, 24.1, 24.4, 24.8, 25.1, 25.8, 25.9, 26.4, and 27.7 degrees two-theta±0.2 degrees two-theta.

6. A crystalline form of voxelotor according to claim 5, which has X-ray powder diffraction pattern substantially as shown in FIG. 3.

7. A crystalline form of voxelotor which is crystalline voxelotor hemi propylene glycol solvate.

8. A crystalline form of voxelotor according to claim 7, wherein the crystalline voxelotor hemi propylene glycol solvate has an X-ray powder diffraction pattern comprising one or more peaks selected from the group consisting of about 8.6, 8.8, 11.3, 12.6, 12.9, 14.5, 15.0, 15.5, 15.6, 16.0, 16.8, 17.1, 17.7, 18.0, 18.6, 19.1, 19.7, 20.2, 20.9, 22.8, 23.1, 23.7, 24.2, 25.1, 25.4, 25.9, 26.7, 27.2, 28.8, 30.3, 31.6, and 32.4 degrees two-theta±0.2 degrees two-theta.

9. A crystalline form of voxelotor according to claim 8, which has an X-ray powder diffraction pattern substantially as shown in FIG. 1.

10. A pharmaceutical composition comprising voxelotor and a pharmaceutically acceptable excipient,
wherein the voxelotor is selected from the group consisting of (i) crystalline voxelotor hemi succinic acid molecular complex, (ii) crystalline voxelotor hemi fumaric acid molecular complex, and (iii) crystalline voxelotor hemi propylene glycol solvate.

11. A method for treating a condition associated with oxygen deficiency in a patient comprising administering a therapeutically effective amount of voxelotor to the patient,
wherein the voxelotor is selected from the group consisting of (i) crystalline voxelotor hemi succinic acid molecular complex, (ii) crystalline voxelotor hemi fumaric acid molecular complex, and (iii) crystalline voxelotor hemi propylene glycol solvate.

12. A method according to claim 11, wherein the condition associated with oxygen deficiency is sickle cell disease.

13. Voxelotor for use in treating a condition associated with oxygen deficiency, wherein the voxelotor is selected from the group consisting of (i) crystalline voxelotor hemi succinic acid molecular complex, (ii) crystalline voxelotor hemi fumaric acid molecular complex, and (iii) crystalline voxelotor hemi propylene glycol solvate.

14. Voxelotor for use in treating a condition according to claim 13, wherein the condition associated with oxygen deficiency is sickle cell disease.
